SEQUENCE LISTING (19 sequences)

SEQ ID NO: 1 - protein, 243 aa, Homo sapiens
ECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 2 - protein, 73 aa, Homo sapiens
ECSQDCATCSYRLVRPADINFLACVMECEGKLPSLKIWETCKELLQLSKPELPQDGTSTLRENSKPEESHLLA

SEQ ID NO: 3 - protein, 5 aa, Homo sapiens
YGGFM

SEQ ID NO: 4 - protein, 5 aa, Homo sapiens
YGGFL

SEQ ID NO: 5 - protein, 20 aa, Homo sapiens
MDELYPMEPEEEANGSEILA

SEQ ID NO: 6 - protein, 41 aa, Homo sapiens
DAEEDDSLANSSDLLKELLETGDNRERSHHQDGSDNEEEVS

SEQ ID NO: 7 - protein, 8 aa, Homo sapiens
YGGFMRGL

SEQ ID NO: 8 - protein, 12 aa, Homo sapiens
SPQLEDEAKELQ

SEQ ID NO: 9 - protein, 11 aa, Homo sapiens
VGRPEWWMDYQ

SEQ ID NO: 10 - protein, 22 aa, Homo sapiens
FAEALPSDEEGESYSKEVPEME

SEQ ID NO: 11 - protein, 29 aa, Homo sapiens
FAEALPSDEEGESYSKEVPEMEKRYGGFM

SEQ ID NO: 12 - protein, 7 aa, Homo sapiens
YGGFMRF

SEQ ID NO: 13 - protein, 8 aa, Homo sapiens
LKELLETG

SEQ ID NO: 14 - protein, 8 aa, Homo sapiens
SDNEEEVS

SEQ ID NO: 15 - protein, 6 aa, Homo sapiens
DAEEDD

SEQ ID NO: 16 - protein, 14 aa, Homo sapiens
EEDDSLANSSDLLK

SEQ ID NO: 17 - protein, 8 aa, Homo sapiens
LKELLETG

SEQ ID NO: 18 - protein, 17 aa, Homo sapiens
TGDNRERSHHQDGSDNE

SEQ ID NO: 19 - protein, 8 aa, Homo sapiens
SDNEEEVS

(19) United States
(12) Patent Application Publication
(10) Pub. No.: US 2022/0291234 A1
(43) Pub. Date: Sep. 15, 2022

(54) A METHOD FOR DIAGNOSING OR MONITORING KIDNEY FUNCTION OR DIAGNOSING KIDNEY DYSFUNCTION IN PEDIATRIC PATIENTS

(71) Applicant: SPHINGOTEC GMBH, Hennigsdorf (DE)

(72) Inventors: Andreas BERGMANN, Berlin (DE); Saskia DE WILDT, Nijmegen (NL)

(73) Assignee: SPHINGOTEC GMBH, Hennigsdorf (DE)

(21) Appl. No.: 17/635,265

(22) PCT Filed: Aug. 14, 2020
(86) PCT No.: PCT/EP2020/072916

§ 371 Date: Feb. 14, 2022

(30) Foreign Application Priority Data
Aug. 15, 2019 (EP) 19191968.7

(51) Int. Cl. G01N 33/68; G16H 50/20; G16H 50/30

(52) U.S. Cl. CPC G01N 33/6893; G16H 50/20; G16H 50/30; G01N 2800/347

(57) ABSTRACT

(a) diagnosing or monitoring kidney function in a subject or (b) diagnosing kidney dysfunction in a subject or (c) predicting or monitoring the risk of an adverse event in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention:
determining the level of Pro-Enkephalin or fragments thereof of at least 5 amino acids in a bodily fluid obtained from said subject; and
correlating said level of Pro-Enkephalin or fragments thereof with kidney function in a subject.

Subject matter of the present invention is a method for (a) diagnosing or monitoring kidney function in a subject or (b) diagnosing kidney dysfunction in a subject or (c) predicting or monitoring the risk of an adverse event in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention comprising determining the level of Pro-Enkephalin or fragments thereof of at least 5 amino acids in a bodily fluid obtained from said subject; and
(a) correlating said level of Pro-Enkephalin or fragments thereof with kidney function in a subject or
(b) correlating said level of Pro-Enkephalin or fragments thereof with kidney dysfunction wherein an elevated level above a certain threshold is predictive or diagnostic for kidney dysfunction in said subject or
(c) correlating said level of Pro-Enkephalin or fragments thereof with said risk of an adverse event in a diseased subject, wherein an elevated level above a certain threshold is predictive for an enhanced risk of said adverse events or
(d) correlating said level of Pro-Enkephalin or fragments thereof with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions,

wherein said Pro-Enkephalin or fragment is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11,

wherein said threshold is in the range of 150-1290 pmol/L,

and wherein said subject is a child.

Further subject-matter of the present invention is a method for diagnosing or monitoring kidney function in a subject comprising:

determining the level of Pro-Enkephalin or fragments thereof of at least 5 amino acids in a bodily fluid obtained from said subject; and

wherein during follow-up measurement, a relative change of Pro-Enkephalin and fragments thereof that is lowered correlates with the improvement of the subject's kidney function, or

wherein during follow-up measurement, a relative change of Pro-Enkephalin and fragment thereof that is increased correlates with the worsening of the subject's kidney function,

wherein said Pro-Enkephalin or fragment thereof is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11; and

wherein said determination of Pro-Enkephalin or fragments thereof of at least 5 amino acids is performed more than once in one patient,

wherein the subject is a child.

Acute kidney injury (AKI) is defined as the abrupt loss of kidney function that results in a decline in glomerular filtration rate (GFR), retention of urea and other nitrogenous waste products, and dysregulation of extracellular volume and electrolytes. The term AKI has largely replaced acute renal failure, as it more clearly defines renal dysfunction as a continuum rather than a discrete finding of failed kidney function. Acute kidney injury (AM) is a frequent and serious complication in critically ill children: the reported incidence is up to 5% in the general ward and up to 35% on pediatric intensive care unit (PICU) (Zwiers et al. 2015. Critical Care 19: 181). Moreover, it has been shown to be an independent risk factor for mortality, prolonged length of ICU-stay and prolonged mechanical ventilation (Alkandari et al. 2011. Crit Care 15: R146). Current consensus criteria for diagnosing AKI are based on changes in serum creatinine (SCr) and urine output (Akcan-Arikan et al. 2007. Kidney International 71: 1028-1035). However, SCr is an indicator of glomerular function rather than renal tubular cell damage, which typically occurs during the initial phase of AKI in ICU patients (Andreoli 2009. Pediatr Nephrol 24:253-63). Moreover, SCr is influenced by factors unrelated to renal function and in the newborn reflects maternal levels immediately after birth (Schwartz and Furth 2007. Pediatr Nephrol 22:1839-1848; Arant 1987. Pediatr Nephrol 1:308-13). In summary, SCr is increasingly considered a late and not very sensitive marker for diagnosing AKI, especially in children. The terms “kidney function” and “renal function” are used synonymously throughout the specification. Likewise, the terms “kidney failure” and “renal failure” are used synonymously throughout the specification. Other proposed biomarkers to detect tubular cell damage are e.g. neutrophil gelatinase-associated lipocalin (NGAL), kidney injury molecule-1 (KIM-1), tissue inhibitor metalloproteinase-2 (TIMP-2) and insulin-like growth factor binding protein-7 (IGFBP-7). However, those markers do not reflect glomerular filtration function and may be affected by comorbidity, inflammation, timing of the measurements and the chosen cut-off values (Kim et al. 2017. Ann Lab Med 37: 388-397; Ostermann et al. 2012. Critical Care 16:233).

The glomerular filtration rate (GFR) can be estimated using a formula with different parameters or determined via a functional biomarker that is filtrated in the glomerulus. As mentioned above, the most commonly used tests to estimate the GFR are based on SCr concentration, although many limitations are acknowledged. Apart from ethnicity, body mass, and sex causing differences in production in creatinine, calculations are influenced by active renal secretion of creatinine (up to 10%-20% of the total clearance) (Shemesh et al. 1985. Kidney Int 285:830-838). This leads to an overestimation of GFR, especially in patients with deteriorating kidney function (Miller and Winkler 1938. J Clin Invest 1938; 171: 31-40). In summary, conventional creatinine-based methods to assess GFR are insensitive, late, and inaccurate.

Plasma clearance of iohexol, an iodine contrast agent that is exclusively filtrated in the glomerulus, has been shown to be equally accurate in determining the GFR as inulin clearance, the current gold standard to measure the true GFR. However, these methods are only frequently used in clinical practice as they are time-consuming and their determination is labour-consuming.

In children formulas by Schwartz et al. using creatinine and height are most frequently used (Schwartz et al. 1987. Pediatr Clin North Am 34:571-590). However, this is not validated for children <1 year and investigated only in small cohorts due to ethical challenges. Moreover, there are wide age intervals used in most pediatric studies and height doesn't correlate to kidney development <1 year. Maturation of renal function is a dynamic process that begins during fetal organogenesis and is complete by early childhood. The developmental increase in the GFR relies on the existence of normal nephrogenesis, a process that begins at 9 weeks of gestation and is complete by 36 weeks of gestation, followed by postnatal changes in renal and intrarenal blood flow. The GFR is approximately 2 to 4 ml per minute per 1.73 m² in term neonates, but it may be as low as 0.6 to 0.8 ml per minute per 1.73 m² in preterm neonates. The GFR increases rapidly during the first two weeks of life and then rises steadily until adult values are reached at 8 to 12 months of age. Similarly, tubular secretion is immature at birth and reaches adult capacity during the first year of life (Boer et al. 2010. Pediatr Nephrol 25:2107-2113; Kearns et al. 2003. N Engl J Med 349:1157-1167). There is a rapid decline of creatinine as kidneys mature in first year and a slight increase at the end of first year due to increased muscle mass leading to higher creatinine production (Boer et al. 2010. Pediatr Nephrol 25:2107-2113).

Proenkephalin A is a precursor of the enkephalin family of endogenous opioids. It is a prohormone that is proteolytically processed to form several active pentapeptides like methionine-enkephalin (Met-Enk) and leucine-enkephalin (Leu-Enk) together with several other peptide fragments (enkelytin and C-terminal extended Met-Enk peptides). In addition to mature enkephalins, other peptides are produced, one of which is a stable proenkephalin peptide 119-159. This peptide fragment's levels in plasma/serum could serve as a surrogate measurement of systemic enkephalin synthesis, because proenkephalin is the predominant source of mature enkephalins. (Ernst et al., 2006. Peptides 27: 1835-1840). Enkephalins are widely secreted to act on locally expressed opioid receptors, specifically the δ opioid receptors. These opioid receptors are also widely expressed, with the highest density found in the kidney (Denning et al. 2008. Peptides 29 (1): 83-921). Subsequent to receptor binding the biological effects of enkephalins include nociception, anesthetics, and cardiovascular regulation (Holaday 1983. Annu. Rev. Pharmacol. Toxicol. 23: 541-594). These δ opioid agonists stimulate natriuresis and diuresis (Sezen et al. 1998. J. Pharmacol. Exp. Ther. 287 (1): 238-245). While several studies have demonstrated that elevated concentrations are associated with adverse outcomes, the association has in general been proportional to the change in renal function. Indeed, increased concentrations are associated with decreased renal function in several populations including sepsis (Marino et al. 2015. J Nephrol 28:717-724), heart failure (Ng et al. 2017. J. Am. Coll. Cardiol. 69 (1): 56-69; Matsue et al. 2017. J. Card. Fail. 23 (3): 231-239), cardiac surgery (Shah et al. 2015. Clin. Nephroi. 83 (1):29-35), and myocardial infarction (Ng et al. 2014. J. Am. Coll. Cardiol. 63 (3) (2014) 280-289).

Plasma Methionine-Enkephalin (Met-Enk) levels and higher molecular weight forms of Met-Enk (C-terminal extended Met-Enk peptides) have been measured in human newborns at birth (Martinez et al. 1991. Biol Neonate 60:102-107). Met-Enk immunoreactivity levels were significantly greater in the newborn infants in comparison to the adult plasma levels with a factor of 15. In contrast, higher molecular weight forms of Met-Enk measured as total Met-Enk immunoreactivity were not statistically different between newborns and adult levels.

It was the surprising finding of the present invention that the levels of Pro-Enkephalin and fragments thereof, especially Pro-Enkephalin 119-159 (MR-PENK, SEQ ID No. 6), are significantly increased in plasma of children compared to adults in healthy status. Moreover, Pro-Enkephalin and fragments thereof, especially Pro-Enkephalin 119-159 (MR-PENK, SEQ ID No. 6), are significantly increased in children with kidney dysfunction when compared to children with normal kidney function.

The terms Pro-Enkephalin, proenkephalin and PENK are used synonymously throughout the specification.

Risk according to the present invention correlates with the risk as defined by the RIFLE criteria. The RIFLE classification consists of three levels of renal dysfunction with increasing severity, namely the “Risk (R)”, “Injury (I)”, and “Failure (F)”, based on the degree of decrease in estimated creatinine clearance (eCCl) and urine output (Table 1). In addition to “R”, “I” and “F”, there are two levels of adverse clinical outcome: “Loss (L)” that refers to persistent renal failure for >4 weeks, and “End-stage (E)” that refers to persistent renal failure for >3 months. The pRIFLE criteria differs from the RIFLE criteria, in that only decrease in eCCl, and not the change in SCr or GFR, is used to determine grading. Furthermore, the eCCl is estimated using the Schwartz formula, which incorporates the height and SCr level of the patient, and an age-adjusted constant (Schwartz et al. 1987. Pediatr Clin North Am 34:571-590), whilst also depending on a longer duration of urine output than in the adult RIFLE classification. Furthermore, there exist additional criteria (AKIN/KDIGO) for pediatrics (Table 1). The KDIGO guidelines refer to pRIFLE for the definition of AKI in children, and the latter remains the one in use for children aged over 1 month (Thomas et al. 2015. Kidney International 87: 62-73).

TABLE 1
pediatric RIFLE Score, AKIN and KDIGO (Thomas et al. 2015. Kidney International 87: 62-73)

| | | AKIN¹⁵/KDIGO^{3, 16} | |
|---|---|---|---|
| Stage | pRIFLE criteria¹²⁶ | Criteria | Urine output |
| Risk or Stage 1 | — | Rise of ≥26 μmol/l^{a} | <0.5 ml/kg/h for more |
| | eGFR decrease by ≥25% | or 0.3 mg/dl within 48 h | than 8 h^{b} |
| | or 50-90% Cr rise | Or 50-99% Cr rise | — |
| | from baseline withing 7 | from baseline within 7 | |
| | days^{c} (1.50-1.99 × baseline) | days^{c} (1.50-1.99 × baseline) | |
| Injury or stage 2 | eGFR decrease by ≥50% | 100-199% Cr rise | <0.5 ml/kg/h for more |
| | or 100-199% Cr rie | from baseline withing 7 | than 16 h^{b} |
| | from baseline within 7 | days^{c} (2.00-2.99 × baseline) | |
| | days^{c} (2.00-2.99 × baseline) | | |
| Failure or stage 3 | eGFR decrease by 75% | ≥200% Cr rise | <0.3 ml/kg/h for 24 h |
| | or eGFR <35 ml/min per | from baseline withing 7 | or anuria for 12 h |
| | 1.73 m² or ≥200% | daysc (≥3.00 × baseline) | — |
| | Cr rise from | Or any requirement for | |
| | baseline within 7 | renal replacement therapy | |
| | days^{c} (≥3.00 × baseline) | | |
| | (In pRIFLE, RRT does | | |
| | not automatically | | |
| | equate to stage Failure | | |
| | or stage 3)¹²⁶ | | |

Abbreviations: AKIN, the AKI Network; Cr, creatinine; eGFR, estimated glomerular filtration rate; KDIGO, Kidney Disease Improving Global Outcomes; RRT, renal replacement therapy.
^{a}Equivalent to 0.3 mg/dl, with the SI units rounded down to the nearest integer.
^{b}Note that the duration of oliguria in the Risk and Injury stages differs from that for the same stage in adults and is quoted for the pRIFLE classification.
^{c}Where the rise is known (based on a prior blood test) or presumed (based on the patient history) to have occurred within 7 days

Subject matter of the present invention is the use of Pro-Enkephalin (PENK) or fragments thereof as marker for kidney function and dysfunction and its clinical utility in healthy and diseased children. Subject matter of the present invention is a method for diagnosing or monitoring kidney function in children or diagnosing kidney dysfunction in children or predicting the risk of adverse events in a diseased child.

A subject of the present invention was also the provision of the prognostic and diagnostic power of PENK or fragments thereof for the diagnosis of kidney function, dysfunction and the prognostic value in diseased children.

Surprisingly, it has been shown that PENK or fragments thereof are powerful and highly significant biomarkers for kidney, its function, dysfunction, risk of adverse events and prognosis and monitoring success of therapy or intervention in children.

According to the present invention said Pro-Enkephalin or fragments thereof is not Leu-Enkephalin and not Met-Enkephalin in one specific embodiment. In another specific embodiment said Pro-Enkephalin fragment is mid-regional Pro-Enkephalin (MR-PENK; SEQ ID No.: 6) or a fragment thereof having at least 5 amino acids.

Subject matter of the present invention is further a method for (a) diagnosing or monitoring kidney function in a subject or (b) diagnosing kidney dysfunction in a subject or (c) predicting or monitoring the risk of an adverse event in a diseased subject wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention comprising
determining the level of immunoreactive analyte by using at least one binder that binds to a region within the amino acid sequence of Pro-Enkephalin (PENK) in a bodily fluid obtained from said subject; and
(a) correlating said level of immunoreactive analyte with kidney function in a subject or
(b) correlating said level of immunoreactive analyte with kidney dysfunction, wherein an elevated level above a certain threshold is predictive or diagnostic for kidney dysfunction in said subject or
(c) correlating said level of immunoreactive analyte with said risk of an adverse event in a diseased subject, wherein an elevated level above a certain threshold is predictive for an enhanced risk of said adverse events or
(d) correlating said level of immunoreactive analyte with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions, and

wherein said threshold is in the range of 150-1290 pmol/L,

wherein said subject is a child.

This means in case a binder is used in the methods of the present invention that binds to a region within the amino acid sequence of Pro-Enkephalin (PENK) in a bodily fluid, then the terms “determining the level of Pro-Enkephalin (PENK) or fragments thereof of at least 5 amino acids in a bodily fluid obtained from said subject” are equivalent to “determining the level of immunoreactive analyte by using at least one binder that binds to a region within the amino acid sequence of Pro-Enkephalin (PENK) in a bodily fluid obtained from said subject”. In a specific embodiment a binder is used in the methods of the present invention that binds to a region within the amino acid sequence of Pro-Enkephalin (PENK) in a bodily fluid. In a specific embodiment said binder used in the methods of the present invention does not bind to a region within the amino acid sequence of leu-enkephalin or met-enkephalin in a bodily fluid. In another specific embodiment of the present invention said at least one binder binds to mid-regional Pro-Enkephalin (MR-PENK) or a fragment thereof having at least 5 amino acids.

The term “subject” as used herein refers to a living human or non-human organism. Preferably herein the subject is a human subject. The subject may be healthy or diseased if not stated otherwise.

The term “child” as used herein refers to a subject that is at the age of 18 years or below, more preferred at the age of 14 years or below, even more preferred at the age of 12 years or below, even more preferred at the age of 8 years or below, even more preferred at the age of 5 years or below, even more preferred at the age of 2 years or below, most preferred at the age of one year or below.

In a specific embodiment said child is a neonate. A neonate refers to a child in the first 28 days after birth and applies to premature, full term, and postmature children.

The term “critically ill patient” is defined as a patient at high risk for actual or potential life-threatening health problems requiring intensive monitoring and care. Those patients may require support for cardiovascular instability (hypertension/hypotension), potentially lethal cardiac arrhythmias, airway or respiratory compromise (such as ventilator support), acute renal failure, or the cumulative effects of multiple organ failure, more commonly referred to now as multiple organ dysfunction syndrome.

In a specific embodiment of the invention it should be understood that those patients may require support for cardiovascular instability (hypertension/hypotension), potentially lethal cardiac arrhythmias, airway or respiratory compromise (such as ventilator support), or the cumulative effects of multiple organ failure, more commonly referred to now as multiple organ dysfunction syndrome.

The term “elevated level” means a level above a certain threshold level. The term “elevated” level may mean a level above a value that is regarded as being a reference level.

The term “diagnosing” in the context of the present invention relates to the recognition and (early) detection of a disease or clinical condition in a subject and may also comprise differential diagnosis.

The term “predicting” in the context of the present invention denotes a prediction of how a subject's (e.g. a patient's) medical condition will progress. This may include an estimation of the chance of recovery or the chance of an adverse outcome for said subject.

The term “monitoring” in the context of the present invention refers to controlling the development of a disease and or pathophysiological condition of a subject.

The term “monitoring the success of a therapy or intervention” in the context of the present invention refers to the control and/or adjustment of a therapeutic treatment of said patient.

Predicting or monitoring the success of a therapy or intervention may be e.g. the prediction or monitoring of success of renal replacement therapy using measurement of Pro-Enkephalin (PENK) or fragments thereof of at least 5 amino acids.

Predicting or monitoring the success of a therapy or intervention may be e.g. the prediction or monitoring of success of treatment with hyaluronic acid in patients having received renal replacement therapy using measurement of Pro-Enkephalin (PENK) or fragments thereof of at least 5 amino acids.

Predicting or monitoring the success of a therapy or intervention may be e.g. the prediction or monitoring of the recovery of renal function in patients with impaired renal function prior to and after renal replacement therapy and/or pharmaceutical interventions using measurement of PENK or fragments thereof of at least 5 amino acids.

A bodily fluid may be selected from the group comprising blood, serum, plasma, urine, cerebrospinal fluid (CSF), and saliva. In one embodiment of the invention the bodily fluid is selected from the group comprising whole blood, plasma, and serum.

Determination of Pro-Enkephalin or fragments thereof exhibit kidney function in the subject. An increased concentration of Pro-Enkephalin or fragments thereof above a certain threshold level indicates a reduced kidney function. During follow up measurements, a relative change of Pro-Enkephalin or fragments thereof correlates with the improvement (lowering Pro-Enkephalin or fragments thereof) and with the worsening (increased Pro-Enkephalin or fragments thereof) of the subjects' kidney function.

Pro-Enkephalin or fragments thereof are diagnostic for kidney dysfunction wherein an elevated level above a certain threshold is predictive or diagnostic for kidney dysfunction in said subject. During follow up measurements, a relative change of Pro-Enkephalin or fragments thereof correlates with the improvement (lowering Pro-Enkephalin or fragments thereof) and with the worsening (increased Pro-Enkephalin or fragments thereof) of the subjects' kidney function.

Pro-Enkephalin or fragments thereof are superior in comparison to other markers for kidney function/dysfunction diagnosis and follow up (NGAL, blood creatinine, creatinine clearance, Cystatin C, Urea). Superiority means higher specificity, higher sensitivity and better correlation to clinical endpoints.

Correlating said level of Pro-Enkephalin or fragments thereof with a risk of an adverse event in a diseased subject (child), wherein an elevated level above a certain threshold is predictive for an enhanced risk of adverse events. In this aspect, Pro-Enkephalin or fragments thereof are superior to above mentioned clinical markers.

Kidney function may be measured by GFR, creatinine clearance, SCr, urinalysis, blood urea nitrogen or urine output. Kidney dysfunction means a reduction of kidney function, e.g. kidney failure.

The diseased subject (child) may suffer or may be at risk to suffer from a disease selected from chronic kidney disease (CKD), acute kidney disease (AKD) or AKI.

Conditions affecting kidney structure and function can be considered acute or chronic, depending on their duration.

AKD is characterized by structural kidney damage for <3 months and by functional criteria that are also found in AKI, or a GFR of <60 ml/min per 1.73 m² for <3 months, or a decrease in GFR by ≥35%, or an increase in serum creatinine (SCr) by >50% for <3 months (Kidney International Supplements, Vol. 2, Issue 1, March 2012, pp. 19-36).

AKI is one of a number of acute kidney diseases and disorders, and can occur with or without other acute or chronic kidney diseases and disorders (Kidney International Supplements, Vol. 2, Issue 1, March 2012, pp. 19-36).

AKI is defined as reduction in kidney function, including decreased GFR and kidney failure. The criteria for the diagnosis of AKI and the stage of severity of AKI are based on changes in SCr and urine output. In AKI no structural criteria are required (but may exist), but an increase in SCr by 50% within 7 days, or an increase by 0.3 mg/dl (26.5 μmol/l), or oliguria is found. AKD may occur in patients with trauma, stroke, sepsis, SIRS, septic shock, respiratory failure, cardiac failure (e.g. acute and post myocardial infarction, heart failure), congenital diaphragmatic hernia, local and systemic bacterial and viral infections, autoimmune diseases, burns, surgery, cancer, liver diseases, lung diseases, as well as in patients receiving nephrotoxins such as calcineurin inhibitors (e.g. cyclosporine), antibiotics (e g aminoglycosides or vancomycin) and anticancer drugs (e.g. cisplatin).

CKD is characterized by a GFR of <60 ml/min per 1.73 m² for >3 months and by kidney damage for >3 months (Kidney International Supplements, 2013; Vol. 3: 19-62).

Kidney failure is a stage of CKD and is defined as a GFR <15 ml/min per 1.73 m² body surface area, or requirement for RRT.

The definitions of AKD, AKI and CKD (according to KDIGO Clinical Practice Guideline for Acute Kidney Injury 2012 Vol 2 (1)) are summarized in Table 2.

TABLE 2
Definition of AKI, AKD and CKD

| | Functional criteria | Structural criteria |
|---|---|---|
| AKI | Increase in SCr by 50% within 7 days, OR | No criteria |
| | Increase in SCr by 0.3 mg/dl (26.5 μmol/l) within 2 | |
| | days, OR | |
| | Oliguria | |
| AKD | AKI, OR | Kidney damage for >3 months |
| | GFR <60 ml/min per 1.73 m² for <3 months, OR | |
| | Decrease in GFR by ≥35% or increase in SCr by >50% | |
| | for <3 months | |
| CKD | GFR <60 ml/min per 1.73 m² for >3 months | Kidney damage for >3 months |
| NKD | GFR ≥60 ml/min per 1.73 m² | No damage |
| | Stable SCr | |

NKD = no kidney disease

In children <2 years the GFR thresholds that delineate and stage kidney damage and reduction in kidney function need to be adapted age-dependently (KDIGO 2012 Clinical Practice Guideline for the Evaluation and Management of Chronic Kidney Disease).

In a preferred embodiment of the invention the diseased subject (child) may suffer from a disease selected from kidney failure, respiratory failure, congenital diaphragmatic hernia, cardiac failure, SIRS, sepsis, septic shock or other critical illness.

The therapy or intervention supporting or replacing kidney function may comprise various methods of renal replacement therapy including but not limited to hemodialysis, peritoneal dialysis, hemofiltration and renal transplantation.

The therapy or intervention supporting or replacing kidney function may also comprise pharmaceutical interventions, kidney-supporting measures as well as adaption and/or withdrawal of nephrotoxic medications.

An adverse event may be selected from the group comprising worsening of kidney function including kidney failure, loss of kidney function and end-stage kidney disease or death due to kidney dysfunction including kidney failure, loss of kidney function and end-stage kidney disease (according to the pediatric RIFLE criteria (Akcan-Arikan et al. 2007. Kidney International 71: 1028-1035)).

In one embodiment of the invention it should be understood that the term fragments of Pro-Enkephalin also include Leu-Enkephalin and Met-Enkephalin.

Subject matter according to the present invention is a method, wherein the level of Pro-Enkephalin or fragments thereof of at least 5 amino acids is determined by using at least one binder to Pro-Enkephalin or fragments thereof of at least 5 amino acids. In one embodiment of the invention said binder is selected from the group comprising an antibody, an antibody fragment or a non-Ig-Scaffold binding to Pro-Enkephalin or fragments thereof of at least 5 amino acids. In a specific embodiment said at least one binder binds to a region with the sequences selected from the group comprising SEQ ID No. 1, 2, 3, 4, 5, 6, 7, 8, 9, 10, 11 and 12. In a specific embodiment said binder do not bind to enkephalin peptides met-enkephalin SEQ ID No: 3, and leu-enkephalin SEQ ID No: 4. In a specific embodiment said at least one binder binds to a region with the sequences selected from the group comprising SEQ ID No. 1, 2, 5, 6, 8, 9, 10 and 11. In another specific embodiment said at least one binder binds to a region with the sequences selected from the group comprising SEQ ID No. 1, 2, 5, 6, 8 and 9. In another very specific embodiment said binder binds to Pro-Enkephalin 119-159, mid-regional Pro-Enkephalin fragment, MR-PENK SEQ ID No. 6.

Pro-Enkephalin has the following sequence:

(Pro-Enkephalin 1-243)
SEQ ID NO. 1
ECSQDCATCSYRLVRPADINFLACVMECEGKLPSLKIWETCKELLQLSKP
ELPQDGTSTLRENSKPEESHLLAKRYGGFMKRYGGFMKKMDELYPMEPEE
EANGSEILAKRYGGFMKKDAEEDDSLANSSDLLKELLETGDNRERSHHQD
GSDNEEEVSKRYGGFMRGLKRSPQLEDEAKELQKRYGGFMRRVGRPEWWM
DYQKRYGGFLKRFAEALPSDEEGESYSKEVPEMEKRYGGFMRF

Fragments of Pro-Enkephalin, that may be determined in a bodily fluid, may be e.g. selected from the group of the following fragments:

SEQ ID NO. 2 (Synenkephalin, Pro-Enkephalin 1-73)
ECSQDCATCSYRLVRPADINFLACVMECEGKLPSLKIWETCKELLQLSKP
ELPQDGTSTLRENSKPEESHLLA
SEQ ID NO. 3 (Met-Enkephalin)
YGGFM
SEQ ID NO. 4 (Leu-Enkephalin)
YGGFL
SEQ ID NO. 5 (Pro-Enkephalin 90-109)
MDELYPMEPEEEANGSEILA
SEQ ID NO 6: (Pro-Enkephalin 119-159, Mid-regional
Pro-Enkephalin-fragment, MR-PENK)
DAEEDDSLANSSDLLKELLETGDNRERSHHQDGSDNEEEVS
SEQ ID NO. 7 (Met-Enkephalin-Arg-Gly-Leu)
YGGFMRGL
SEQ ID NO. 8 (Pro-Enkephalin 172-183)
SPQLEDEAKELQ
SEQ ID NO. 9 (Pro-Enkephalin 193-203)
VGRPEWWMDYQ
SEQ ID NO. 10 (Pro-Enkephalin 213-234)
FAEALPSDEEGESYSKEVPEME
SEQ ID NO. 11 (Pro-Enkephalin 213-241)
FAEALPSDEEGESYSKEVPEMEKRYGGFM
SEQ ID NO. 12 (Met-Enkephalin-Arg-Phe)
YGGFMRF

Determining the level of Pro-Enkephalin including Leu-Enkephalin and Met-Enkephalin or fragments thereof may mean that the immunoreactivity towards Pro-Enkephalin or fragments thereof including Leu-Enkephalin and Met-Enkephalin is determined. A binder used for determination of Pro-Enkephalin including Leu-Enkephalin and Met-Enkephalin or fragments thereof depending of the region of binding may bind to more than one of the above displayed molecules. This is clear to a person skilled in the art.

Thus, according to the present invention the level of immunoreactive analyte by using at least one binder that binds to a region within the amino acid sequence of any of the above peptides and peptide fragments, (i.e. Pro-Enkephalin (PENK) and fragments according to any of the sequences 1 to 12), is determined in a bodily fluid obtained from said subject; and correlated to the specific embodiments of clinical relevance.

In a more specific embodiment of the method according to the present invention the level of MR-PENK is determined (SEQ ID NO. 6: Pro-Enkephalin 119-159, Mid-regional Pro-Enkephalin-fragment, MR-PENK). In a more specific embodiment, the level of immunoreactive analyte by using at least one binder that binds to MR-PENK is determined and is correlated to the above-mentioned embodiments according to the invention to the specific embodiments of clinical relevance, e.g.
(a) correlating said level of immunoreactive analyte with kidney function in a subject or
(b) correlating said level of immunoreactive analyte with kidney dysfunction wherein an elevated level above a certain threshold is predictive or diagnostic for kidney dysfunction in said subject or
(c) correlating said level of immunoreactive analyte with said risk of an adverse event in a diseased subject, wherein an elevated level above a certain threshold is predictive for an enhanced risk of said adverse events or
(d) correlating said level of immunoreactive analyte with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions,
wherein said threshold is in the range of 150-1290 pmol/L,
wherein said subject is a child.

Alternatively, the level of any of the above analytes may be determined by other analytical methods e.g. mass spectroscopy.

Thus, subject matter of the present invention is method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention comprising
determining the level of immunoreactive analyte by using at least one binder that binds to a region within the amino acid sequence of a peptide selected from the group comprising the Pro-Enkephalin peptides and fragments of SEQ ID No. 1 to 12 in a bodily fluid obtained from said subject; and
(a) correlating said level of Pro-Enkephalin or fragments thereof with kidney function in a subject or
(b) correlating said level of Pro-Enkephalin or fragments thereof with kidney dysfunction wherein an elevated level above a certain threshold is predictive or diagnostic for kidney dysfunction in said subject or
(c) correlating said level of Pro-Enkephalin or fragments thereof with said risk of an adverse event in a diseased subject, wherein an elevated level above a certain threshold is predictive for an enhanced risk of said adverse events or
(d) correlating said level of Pro-Enkephalin or fragments thereof with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions,
wherein said threshold is in the range of 150-1290 pmol/L,
wherein said subject is a child.

In a specific embodiment the level of immunoreactive analyte is determined by using at least one binder that binds to a region within the amino acid sequence of a peptide selected from the group comprising Pro-Enkephalin or fragments thereof of at least 5 amino acids. In a specific embodiment said at least one binder binds to a region with the sequences selected from the group comprising SEQ ID No. 1, 2, 3, 4, 5, 6, 7, 8, 9, 10, 11 and 12. In a specific embodiment said binder do not bind to enkephalin peptides Met-Enkephalin SEQ ID No: 3, and Leu-Enkephalin SEQ ID No: 4. In a specific embodiment said at least one binder binds to a region with the sequences selected from the group comprising SEQ ID No. 1, 2, 5, 6, 8, 9, 10 and 11. In another specific embodiment said at least one binder binds to a region with the sequences selected from the group comprising SEQ ID No. 1, 2, 5, 6, 8 and 9. In another very specific embodiment said binder binds to Pro-Enkephalin 119-159, Mid-regional Pro-Enkephalin-fragment, MR-PENK (SEQ ID No. 6). The before mentioned binder binds to said peptides in a bodily fluid obtained from said subject.

In one embodiment of the invention said binder is selected from the group comprising an antibody, an antibody fragment or a non-Ig-Scaffold binding to Pro-Enkephalin or fragments thereof of at least 5 amino acids.

In a specific embodiment the level of Pro-Enkephalin or fragments thereof are measured with an immunoassay using antibodies or fragments of antibodies binding to Pro-Enkephalin or fragments thereof. An immunoassay that may be useful for determining the level of Pro-Enkephalin or fragments thereof of at least 5 amino acids may comprise the steps as outlined in Example 1. All thresholds and values have to be seen in correlation to the test and the calibration used according to Example 1. A person skilled in the art may know that the absolute value of a threshold might be influenced by the calibration used. This means that all values and thresholds given herein are to be understood in context of the calibration used in herein (Example 1).

According to the invention the diagnostic binder to Pro-Enkephalin is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH₃ domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines.

In a specific embodiment the level of Pro-Enkephalin or fragments thereof are measured with an assay using binders selected from the group comprising aptamers, non-Ig scaffolds as described in greater detail below binding to Pro-Enkephalin or fragments thereof.

Binder that may be used for determining the level of Pro-Enkephalin or fragments thereof exhibit an affinity constant to Pro-Enkephalin of at least 10⁷ M⁻¹, preferred 10⁸ M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention. Binding affinity may be determined using the Biacore method, offered as service analysis e.g. at Biaffin, Kassel, Germany (http://www.biaffin.com/de/).

In addition to antibodies other biopolymer scaffolds are well known in the art to complex a target molecule and have been used for the generation of highly target specific biopolymers. Examples are aptamers, spiegelmers, anticalins and conotoxins. Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds (e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

The threshold level is a level, which allows for allocating the subject into a group of subjects who are diagnosed as having kidney disease/dysfunction or being at risk of an adverse event, or into a group of subjects who are not diagnosed as having kidney disease/dysfunction or being at risk of an adverse event. Thus, the threshold level shall allow for differentiating between a subject who is diagnosed as having kidney disease/dysfunction or being at risk of an adverse event, or into a group of subjects who are not diagnosed as having kidney disease/dysfunction or being at risk of an adverse event. It is known in the art how threshold levels can be determined. Threshold levels are predetermined values and are set to meet routine requirements in terms of e.g. specificity and/or sensitivity. These requirements can vary. It may for example be that sensitivity or specificity, respectively, has to be set to certain limits, e.g. 80%, 90%, 95% or 98%, respectively.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “reference group” (i.e. apparently healthy and/or without signs and symptoms of kidney failure) and “disease” populations (i.e. patients suffering from renal failure). For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “reference” group, and a ROC curve created. These methods are well known in the art (See, e.g., Hanley et al. 1982. Radiology 143: 29-36). Preferably, ROC curves result in an AUC of greater than about 0.5, more preferably greater than about 0.7, still more preferably greater than about 0.8, even more preferably greater than about 0.85, and most preferably greater than about 0.9. The term “about” in this context refers to +/−5% of a given measurement.

A reference group may be a healthy population, e.g. with no signs and symptoms of a disease. In a further aspect of the invention, a reference group may be a population of subjects suffering from a disease or disorder, in particular non-critical diseases or interventions therefor (e.g. inguinal hernia repair, orthopaedic surgery, bronchoscopy, hyperbilirubinemia, sleep apnea test) or critical diseases (e.g. respiratory failure, congenital diaphragmatic hernia, cardiac failure, SIRS, sepsis, septic shock or other critical illness) without signs and symptoms of kidney dysfunction or worsening of kidney function. A reference group may consist of more than one reference subjects.

The horizontal axis of the ROC curve represents (1-specificity), which increases with the rate of false positives. The vertical axis of the curve represents sensitivity, which increases with the rate of true positives. Thus, for a particular cut-off threshold selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

Threshold levels can further be obtained for instance from a Kaplan-Meier analysis, where the occurrence of a disease is correlated with the quartiles of the cardiovascular markers in the population. According to this analysis, subjects with cardiovascular marker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred threshold values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the threshold value depending on whether it is considered more appropriate to identify most of the subjects at risk at the expense of also identifying “false positives”, or whether it is considered more appropriate to identify mainly the subjects at high risk at the expense of missing several subjects at moderate risk.

For example, the 75^{th} percentile, more preferred the 90^{th} percentile, even more preferred a 95^{th} percentile, most preferred the 99^{th} percentile values can be used for the upper limits of the normal range.

The threshold level may vary depending on various physiological parameters such as age, gender or sub-population, as well as on the means used for the determination of Pro-Enkephalin and fragments thereof referred to herein.

In a specific embodiment of the invention, said threshold levels are age-dependent. As shown in the examples, the values for MR-PENK revealed the use of more than one threshold value depending on the age of the subject. The threshold values decreased with increasing age of the subjects.

In one embodiment of the invention, the subjects can be divided into age groups and a specific threshold is assigned to each of these age-groups.

In one embodiment of the invention, the threshold for Pro-Enkephalin or fragments thereof in a child is in the range of 150-1290 pmol/L.

The threshold for Pro-Enkephalin or fragments thereof may be grouped for particular age intervals. Alternatively, continuous thresholds may be applied for the respective age of the children. For example, the threshold may be set for children at the age interval of one year or below between 250 and 1000 pmol/, preferably between 400 and 650 pmol/L.

In one specific embodiment the level of Pro Enkephalin or fragments thereof is measured with an immunoassay and said binder is an antibody, or an antibody fragment binding to Pro-Enkephalin or fragments thereof of at least 5 amino acids.

In one specific embodiment the assay used comprises two binders that bind to two different regions within the region of Pro-Enkephalin that is amino acid 133-140 (LKELLETG, SEQ ID No. 13) and amino acid 152-159 (SDNEEEVS, SEQ ID No. 14), wherein each of said regions comprises at least 4 or 5 amino acids.

In one embodiment of the invention the assays for determining Pro-Enkephalin or fragments in a sample are able to quantify the Pro-Enkephalin or Pro-Enkephalin fragments of healthy children and is <15 pmol/L, preferably <10 pmol/L and most preferred <6 pmol/L.

Subject matter of the present invention is the use of at least one binder that binds to a region within the amino acid sequence of a peptide selected from the group comprising the peptides and fragments of SEQ ID No. 1 to 12 in a bodily fluid obtained from said subject in a method a for (a) diagnosing or monitoring kidney function in subject or (b) diagnosing kidney dysfunction in a subject or (c) predicting or monitoring the risk of an adverse events in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention, wherein said subject is a child.

In one embodiment of the invention said binder is selected from the group comprising an antibody, an antibody fragment or a non-Ig scaffold binding to Pro-Enkephalin or fragments thereof of at least 5 amino acids. In a specific embodiment said at least one binder binds to a region with the sequences selected from the group comprising SEQ ID No. 1, 2, 3, 4, 5, 6, 7, 8, 9, 10, 11 and 12. In a specific embodiment said binder do not bind to enkephalin peptides met-enkephalin (SEQ ID No: 3), and leu-enkephalin (SEQ ID No: 4). In a specific embodiment said at least one binder binds to a region with the sequences selected from the group comprising SEQ ID No. 1, 2, 5, 6, 8, 9, 10 and 11. In another specific embodiment said at least one binder binds to a region with the sequences selected from the group comprising SEQ ID No. 1, 2, 5, 6, 8 and 9. In another very specific embodiment said binder binds to Pro-Enkephalin 119-159, mid-regional Pro-Enkephalin-fragment, MR-PENK (SEQ ID No. 6).

In a more specific embodiment the at least one binder binds to a region within the amino acid sequence of Pro-Enkephalin 119-159, mid-regional Pro-Enkephalin fragment, MR-PENK (SEQ ID No. 6) in a bodily fluid obtained from said subject, more specifically to amino acid 133-140 (LKELLETG, SEQ ID No. 13) and/or amino acid 152-159 (SDNEEEVS, SEQ ID No. 14), wherein each of said regions comprises at least 4 or 5 amino acids.

Thus, according to the present methods the level of immunoreactivity of the above binder is determined in a bodily fluid obtained from said subject. Level of immunoreactivity means the concentration of an analyte determined quantitatively, semi-quantitatively or qualitatively by a binding reaction of a binder to such analyte, where preferably the binder has an affinity constant for binding to the analyte of at least 10⁸ M⁻¹, and the binder may be an antibody or an antibody fragment or a non-Ig scaffold, and the binding reaction is an immunoassay.

The present methods using PENK and fragments thereof, especially MR-PENK, are far superior over the methods and biomarkers used according to the prior art for (a) diagnosing or monitoring kidney function in a subject or (b) diagnosing kidney dysfunction in a subject or (c) predicting or monitoring the risk of an adverse event in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention.

Subject of the present invention is also a method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention supporting or replacing kidney function comprising various methods of renal replacement therapy including but not limited to hemo-dialysis, peritoneal dialysis, hemofiltration and renal transplantation according to any of the preceding embodiments, wherein the level of pro-Enkephalin or fragments thereof of at least 5 amino acids in a bodily fluid obtained from said subject either alone or in conjunction with other prognostically useful laboratory or clinical parameters is used which may be selected from the following alternatives:
Comparison with the median of the level of Pro-Enkephalin or fragments thereof of at least 5 amino acids in a bodily fluid obtained from said subject in an ensemble of pre-determined samples in a population of “healthy” or “apparently healthy” subjects,
Comparison with a quantile of the level of Pro-Enkephalin or fragments thereof of at least 5 amino acids in a bodily fluid obtained from said subject in an ensemble of pre-determined samples in a population of “healthy” or “apparently healthy” subjects,
Calculation based on Cox Proportional Hazards analysis or by using Risk index calculations such as the NRI (Net Reclassification Index) or the IDI (Integrated Discrimination Index),

wherein said subject is a child.

Said additionally at least one clinical parameter that may be determined is selected from the group comprising: beta-trace protein (BTP), cystatin C, KIM-1, TIMP-2, IGFBP-7, blood urea nitrogen (BUN), NGAL, Creatinine Clearance, serum Creatinine (SCr), urea Pediatric Risk of Mortality III [PRISM-III] score, Pediatric Index of Mortality 2 [PIM-II] score and Apache Score.

In one embodiment of the invention said method is performed more than once in order to monitor the function or dysfunction or risk of said subject or in order to monitor the course of treatment of kidney and/or disease. In one specific embodiment said monitoring is performed in order to evaluate the response of said subject to preventive and/or therapeutic measures taken.

In one embodiment of the invention the method is used in order to stratify said subjects into risk groups.

Subject matter of the invention is further an assay for determining Pro-Enkephalin and Pro-Enkephalin fragments in a sample comprising two binders that bind to two different regions within the region of Pro-Enkephalin that is amino acid 133-140 (LKELLETG, SEQ ID NO. 13) and amino acid 152-159 (SDNEEEVS, SEQ ID NO. 14), wherein each of said regions comprises at least 4 or 5 amino acids.

In one embodiment of the invention it may be a so-called POC-test (point-of-care), that is a test technology which allows performing the test within less than 1 hour near the patient without the requirement of a fully automated assay system. One example for this technology is the immunochromatographic test technology.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In one embodiment of the invention at least one of said two binders is labeled in order to be detected.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person.

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluorescein-isothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxy-rhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarins such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Chemiluminescent label may be acridinium ester label, steroid labels involving isoluminol labels and the like. Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “binder molecules” are molecules which may be used to bind target molecules or molecules of interest, i.e. analytes (i.e. in the context of the present invention PENK and fragments thereof), from a sample. Binder molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acidic phosphatase, glucose-6-phosphate dehydrogenase, horse radish peroxidase (HRP) and so on.

In one embodiment of the invention at least one of said two binders is bound to a solid phase as magnetic particles, and polystyrene surfaces.

In one embodiment of the assays for determining Pro-Enkephalin or Pro-Enkephalin fragments in a sample according to the present invention such assay is a sandwich assay, preferably a fully automated assay. It may be an ELISA fully automated or manual. It may be a so-called POC-test (point-of-care). Examples of automated or fully automated assay comprise assays that may be used for one of the following systems: Roche Elecsys®, Abbott Architect®, Siemens Centauer®, Brahms Kryptor®, Biomerieux Vidas®, Alere Triage®, Ortho Vitros®. Examples of test formats are provided above.

In one embodiment of the assays for determining Pro-Enkephalin or Pro-Enkephalin fragments in a sample according to the present invention at least one of said two binders is labeled in order to be detected. Examples of labels are provided above.

In one embodiment of the assays for determining Pro-Enkephalin or Pro-Enkephalin fragments in a sample according to the present invention at least one of said two binders is bound to a solid phase. Examples of solid phases are provided above.

In one embodiment of the assays for determining Pro-Enkephalin or Pro-Enkephalin fragments in a sample according to the present invention said label is selected from the group comprising chemiluminescent label, enzyme label, fluorescence label, radioiodine label. A further subject of the present invention is a kit comprising an assay according to the present invention wherein the components of said assay may be comprised in one or more container.

In one embodiment subject matter of the present invention is a point-of-care device for performing a method according to the invention, wherein said point-of-care device comprises at least one antibody or antibody fragment directed to either amino acid 133-140 (LKELLETG, SEQ ID No. 13) or amino acid 152-159 (SDNEEEVS, SEQ ID NO. 14), wherein each of said regions comprises at least 4 or 5 amino acids.

In one embodiment subject matter of the present invention is a point-of-care device for performing a method according to the invention, wherein said point-of-care device comprises at least two antibodies or antibody fragments directed to amino acid 133-140 (LKELLETG, SEQ ID No. 13) and amino acid 152-159 (SDNEEEVS, SEQ ID No. 14), wherein each of said regions comprises at least 4 or 5 amino acids.

In one embodiment subject matter of the present invention is a kit or performing a method according to the invention, wherein said point-of-care device comprises at least one antibody or antibody fragment directed to either amino acid 133-140 (LKELLETG, SEQ ID No. 13) or amino acid 152-159 (SDNEEEVS, SEQ ID No. 14), wherein each of said regions comprises at least 4 or 5 amino acids.

In one embodiment subject matter of the present invention is a kit for performing a method according to the invention, wherein said point-of-care device comprises at least two antibodies or antibody fragments directed to amino acid 133-140 (LKELLETG, SEQ ID No. 13) and amino acid 152-159 (SDNEEEVS, SEQ ID No. 14), wherein each of said regions comprises at least 4 or 5 amino acids.

With the above context, the following consecutively numbered embodiments provide further specific aspects of the invention:
1. A method for (a) diagnosing or monitoring kidney function in a subject or (b) diagnosing kidney dysfunction in a subject or (c) predicting or monitoring the risk of an adverse event in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention comprising
determining the level of Pro-Enkephalin or fragments thereof of at least 5 amino acids in a bodily fluid obtained from said subject; and
   (a) correlating said level of Pro-Enkephalin or fragments thereof with kidney function in a subject or
   (b) correlating said level of Pro-Enkephalin or fragments thereof with kidney dysfunction, wherein an elevated level above a certain threshold is predictive or diagnostic for kidney dysfunction in said subject or
   (c) correlating said level of Pro-Enkephalin or fragments thereof with said risk of an adverse event in a diseased subject, wherein an elevated level above a certain threshold is predictive for an enhanced risk of said adverse events or
   (d) correlating said level of Pro-Enkephalin or fragments thereof with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions, wherein said Pro-Enkephalin or fragment is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11,
wherein said threshold is in the range of 150-1290 pmol/L,
and wherein said subject is a child.
2. A method for diagnosing or monitoring kidney function in a subject comprising:
   determining the level of Pro-Enkephalin or fragments thereof of at least 5 amino acids in a bodily fluid obtained from said subject; and
   wherein during follow-up measurement, a relative change of Pro-Enkephalin and fragments thereof that is lowered correlates with the improvement of the subject's kidney function, or
   wherein during follow-up measurement, a relative change of Pro-Enkephalin and fragment thereof that is increased correlates with the worsening of the subject's kidney function,
   wherein said Pro-Enkephalin or fragment thereof is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11; and
   wherein said determination of Pro-Enkephalin or fragments thereof of at least 5 amino acids is performed more than once in one patient, wherein the subject is a child.
3. A method according to embodiment 1 and 2, wherein said subject is a child at the age of 18 years or below, more preferred at the age of 14 years or below, even more preferred at the age of 12 years or below, even more preferred at the age of 8 years or below, even more preferred at the age of 5 years or below, even more preferred at the age of 2 years or below, most preferred at the age of one year or below.
4. A method according to embodiment 1 to 3, wherein said child is critically ill.
5. A method according to any of embodiments 1 to 4 comprising
   determining the level of immunoreactive analyte by using at least one binder that binds to a region within the amino acid sequence of Pro-Enkephalin (PENK) or fragments thereof in a bodily fluid obtained from said subject; and
   (a) correlating said level of immunoreactive analyte with kidney function in a subject or
   (b) correlating said level of immunoreactive analyte with kidney dysfunction wherein an elevated level above a certain threshold is predictive or diagnostic for kidney dysfunction in said subject or
   (c) correlating said level of immunoreactive analyte with said risk of an adverse event in a diseased subject, wherein an elevated level above a certain threshold is predictive for an enhanced risk of said adverse events or
   (d) correlating said level of immunoreactive analyte with success of a therapy or intervention in a diseased subject, wherein a level below a certain threshold is predictive for a success of therapy or intervention.
6. A method according to any of the embodiments 1 to 5, wherein said at least one binder binds to a region within the amino acid sequence selected from the group comprising SEQ ID No. 1, 2, 5, 6, 8, 9, 10 and 11, preferably said at least one binder binds to a region with the sequences selected from the group comprising SEQ ID No. 1, 2, 5, 6, 8 and 9, preferably said at least one binder binds to SEQ ID No. 6.
7. A method according to any of the preceding embodiments, wherein the level of Pro-Enkephalin is measured with an immunoassay and said binder is an antibody, or an antibody fragment binding to Pro-Enkephalin or fragments thereof of at least 5 amino acids.
8. A method according to any of the embodiments 1 to 7, wherein an assay is used comprising two binders that bind to two different regions within the region of Pro-Enkephalin that is amino acid 133-140 (LKELLETG, SEQ ID NO. 13) and amino acid 152-159 (SDNEEEVS, SEQ ID No. 14), wherein each of said regions comprises at least 4 or 5 amino acids.
9. A method according to any of embodiments 1 to 8, wherein an assay is used for determining the level of Pro-Enkephalin or fragments thereof of at least 5 amino acids and wherein the assay sensitivity of said assay is able to quantify the Pro-Enkephalin or Pro-Enkephalin fragments of healthy subjects and is <15 pmol/L.
10. A method according to any of embodiments 1 to 9 wherein said bodily fluid may be selected from the group comprising whole blood, serum, plasma, urine, cerebrospinal liquid (CSF), and saliva.
11. A method according to embodiments 1 to 10, wherein additionally at least one clinical parameter is determined selected from the group comprising: beta-trace protein (BTP), cystatin C, KIM-1, TIMP-2, IGFBP-7, blood urea nitrogen (BUN), NGAL, Creatinine Clearance, serum Creatinine (SCr), urea, Pediatric Risk of Mortality III [PRISM-III] score, Pediatric Index of Mortality 2 [PIM-II] score and Apache Score.
12. A method according to any of embodiments 1 to 11, wherein said determination of Pro-Enkephalin or fragments thereof of at least 5 amino acids is performed more than once in one patient.
13. A method according to any of embodiments 1 to 12, wherein said monitoring is performed in order to evaluate the response of said subject to preventive and/or therapeutic measures taken.
14. A method according to any of embodiments 1 to 13 in order to stratify said subjects into risk groups.
15. A point-of-care device for performing a method according to any of embodiments 1 to 14, wherein said point of care device comprises at least two antibodies or antibody fragments directed to amino acid 133-140 (LKELLETG, SEQ ID No. 13) and amino acid 152-159 (SDNEEEVS, SEQ ID No. 14).
16. A kit for performing a method according to any of embodiments 1 to 15, wherein said kit comprises at least two antibodies or antibody fragments directed to amino acid 133-140 (LKELLETG, SEQ ID No. 13) and amino acid 152-159 (SDNEEEVS, SEQ ID No. 14).

### EXAMPLES

### Example 1

Development of Antibodies

Peptides

Peptides were synthesized (JPT Technologies, Berlin, Germany).

Peptides/conjugates for Immunization:

Peptides for immunization (Table 3) were synthesized (JPT Technologies, Berlin, Germany) with an additional N-terminal Cysteine residue for conjugation of the peptides to bovine serum albumin (BSA). The peptides were covalently linked to BSA by using Sulfo-SMCC (Perbio Science, Bonn, Germany). The coupling procedure was performed according to the manual of Perbio.

TABLE 3
immunization peptides and antibody names

| Peptide for | Pro-Enkephalin- | |
|---|---|---|
| immunization | sequence | Antibody name |
| (C)DAEEDD | 119-125 | NT-MR-PENK |
| (C)EEDDSLANSSDLLK | 121-134 | NM-MR-PENK |
| (C)LKELLETG | 133-140 | MR-MR-PENK |
| (C)TGDNRERSHHQDGSDNE | 139-155 | MC-MR-PENK |
| (C)SDNEEEVS | 152-159 | CT-MR-PENK |

The antibodies were generated according to the following method:

A BALB/c mouse was immunized with 100 μg peptide-BSA-conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intravenous injection.

Splenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After retesting the selected cultures were cloned and re-cloned using the limiting-dilution technique and the isotypes were determined.

(Lane, R. D. 1985 J. Immunol. Meth. 81: 223-228; Ziegler, B. et al. 1996. Horm. Metab. Res. 28: 11-15).

Monoclonal Antibody Production

Antibodies were produced via standard antibody production methods (Marx et al. 1997. ATLA 25, 121) and purified via Protein A-chromatography. The antibody purities were >95% based on SDS gel electrophoresis analysis.

Labelling and Coating of Antibodies.

All antibodies were labelled with acridinium ester according the following procedure:

Labelled compound (tracer): 100 μg (100 μl) antibody (1 mg/ml in PBS, pH 7.4), was mixed with 10 μl Acridinium NHS-ester (1 mg/ml in acetonitrile, InVent GmbH, Germany) (EP 0353971) and incubated for 20 min at room temperature. Labelled antibody was purified by gel-filtration HPLC on Bio-Sil SEC 400-5 (Bio-Rad Laboratories, Inc., USA) The purified labelled antibody was diluted in (300 mmol/l potassium phosphate, 100 mmol/l NaCl, 10 mmol/l Na-EDTA, 5 g/l bovine serum albumin, pH 7.0). The final concentration was approx. 800.000 relative light units (RLU) of labelled compound (approx. 20 ng labelled antibody) per 200 Acridiniumester chemiluminescence was measured by using an AutoLumat LB 953 (Berthold Technologies GmbH & Co. KG).

Solid phase antibody (coated antibody): Polystyrene tubes (Greiner Bio-One International AG, Austria) were coated (18 hat room temperature) with antibody (1.5 μg antibody/0.3 ml 100 mmol/1 NaCl, 50 mmol/l Tris/HCl, pH 7.8). After blocking with 5% bovine serum albumin, the tubes were washed with PBS, pH 7.4 and vacuum dried.

Antibody Specificity

Antibody cross-reactivities were determined as follows: 1 μg peptide in 300 μl PBS, pH 7.4 was pipetted into Polystyrene tubes and incubated for 1 h at room temperature. After incubation the tubes were washed 5 times (each 1 ml) using 5% BSA in PBS, pH 7.4. Each of the labelled antibodies were added (300 μl in PBS, pH 7.4, 800.000 RLU/300 μl) an incubated for 2 h at room temperature, After washing 5 times (each 1 ml of washing solution (20 mmol/l PBS, pH 7.4, 0.1% Triton X 100), the remaining luminescence (labelled antibody) was quantified using the AutoLumat Luminometer 953. Synthetic MR-PENK peptide was used as reference substance (100%).

The cross-reactivities of the different antibodies are listed in table 4.

TABLE 4
cross-reactivities of the different
PENK-antibodies

| | | | | TGDNRE | | MR-PENK |
|---|---|---|---|---|---|---|
| | DAEE | EEDDSLA | LKELL | RSHHQD | SDNE | (SEQ ID |
| Antibody | DD | NSSDLLK | ETG | GSDNE | EEVS | NO. 6) |
| NT-MR- | 121 | 10 | <1 | <1 | <1 | 100 |
| PENK | | | | | | |
| NM-MR- | <1 | 98 | <1 | <1 | <1 | 100 |
| PENK | | | | | | |
| MR-MR- | <1 | <1 | 105 | <1 | <1 | 100 |
| PENK | | | | | | |
| MC-MR- | <1 | <1 | <1 | 115 | <1 | 100 |
| PENK | | | | | | |
| CT-MR- | <1 | <1 | <1 | <1 | 95 | 100 |
| PENK | | | | | | |

All antibodies bound the MR-PENK peptide, comparable to the peptides which were used for immunization. Except for NT-MR-PENK-antibody (10% cross reaction with EEDDSLANSSDLLK), no antibody showed a cross reaction with MR-PENK fragments not used for immunization of the individual antibody.

Pro-Enkephalin Immunoassay:

50 μl of sample (or calibrator) was pipetted into coated tubes, after adding labelled antibody (200 ul), the tubes were incubated for 2 h at 18-25° C. Unbound tracer was removed by washing 5 times (each 1 ml) with washing solution (20 mmol/l PBS, pH 7.4, 0.1% Triton X-100). Tube-bound labelled antibody was measured by using the Luminometer 953. Using a fixed concentration of 1000 pmol/of MR-PENK. The signal (RLU at 1000 pmol MR-PENK/l) to noise (RLU without MR-PENK) ratio of different antibody combinations is given in table 5. All antibodies were able to generate a sandwich complex with any other antibody. Surprisingly, the strongest signal to noise ratio (best sensitivity) was generated by combining the MR-MR-PENK- and CT-MR-PENK antibody. Subsequently, we used this antibody combination to perform the MR-PENK-immunoassay for further investigations. MR-MR-PENK antibody was used as coated tube antibody and CT-MR-PENK antibody was used as labelled antibody.

TABLE 5
signal to noise ratio of different antibody combinations

| | | | | | |
|---|---|---|---|---|---|
| Solid phase | NT-MR- | NM-MR- | MR-MR- | MC-MR- | CT-MR- |
| antibody | PENK | PENK | PENK | PENK | PENK |

| | | | | | |
|---|---|---|---|---|---|
| Labelled | | | | | |
| antibody | | | | | |
| NT-MR- | / | 27 | 212 | 232 | <1 |
| PENK | | | | | |
| NM-MR- | 36 | / | 451 | 487 | <1 |
| PENK | | | | | |
| MR-MR- | 175 | 306 | / | 536 | 1050 |
| PENK | | | | | |
| MC-MR- | 329 | 577 | 542 | / | <1 |
| PENK | | | | | |
| CT-MR- | <1 | 615 | 1117 | 516 | / |
| PENK | | | | | |

Calibration:

The assay was calibrated, using dilutions of synthetic MR-PENK, diluted in 20 mM K₂PO₄, 6 mM EDTA, 0.5% BSA, 50 μM Amastatin, 100 μM Leupeptin, pH 8.0. FIG. 1 shows a typical Pro-Enkephalin dose/signal curve. The assay sensitivity was 20 determinations of calibrator zero (no addition of MR-PENK)+2SD) 5.5 pmol/L.

### Example 2

Reference values and performance of Proenkephalin A 119-159 as a biomarker for acute kidney injury in children under one year of age.

### INTRODUCTION

Acute kidney injury (AKI) is common in hospitalized children, with prevalences in the pediatric ward and pediatric intensive care unit (PICU) ranging from 5-51%¹⁻³. AKI is independently associated with morbidity and mortality, which appears partially due to accumulation of (toxic) solutes in plasma, including renally excreted drugs⁴⁻⁶.

In most PICUs AKI is diagnosed using serum creatinine concentration (SCr) as a surrogate marker for glomerular filtration rate (GFR). Although SCr provides an inexpensive, minimally invasive and fast estimation of GFR it has several drawbacks as a biomarker for GFR in young children: it shows a delayed increase following AKI, may overestimate GFR due to tubular secretion, production depends on muscle mass and it reflects maternal GFR in the first days of life due to placental transfer⁷⁻¹⁰.

Other biomarkers, like cystatin C (CysC) and β-trace protein (BTP), accurately estimate GFR as functional markers¹¹⁻¹³. CysC detects AKI in critically ill patients up to two days earlier than SCr¹⁴, but concentrations are influenced by inflammation and age^{15,16}. BTP is less influenced by age, race or gender, but is less accurate than other biomarkers^{11,17}. In addition to these functional biomarkers, urinary biomarkers of tubular damage like neutrophil gelatinase-associated lipocalin (NGAL) and kidney injury molecule-1 (KIM-1) have been proposed for the rapid detection of AKI but both are influenced by several pathophysiological factors^{14, 15, 18}. Hence, these biomarkers all present limitations to serve as accurate, endogenous and robust biomarkers for the diagnosis of AKI.

Proenkephalin A 119-159 (PENK) is an endogenous, monomeric peptide cleaved from preproenkephalin A, together with enkephalin peptides¹⁹. Enkephalins bind to opioid receptors and are produced in the central nervous system, kidney, muscles, lung, intestine and heart²⁰. Since the kidneys possess the highest density of opioid receptors, enkephalins are implicated in regulation of kidney function²¹. In addition, PENK possesses several characteristics for an ideal GFR biomarker: it is endogenous, freely filtered by glomeruli due to its small molecular size (4.5 kDa), has no known tubular handling or extra-renal clearance, not bound to plasma proteins and shows stable production in various disease states, independent of inflammation and other non-renal factors²².

To date, research on PENK in healthy and diseased adults support PENK as a marker of kidney function; PENK is an early indicator of AKI, independently predicts future impairment of kidney function and shows high correlations with estimated and measured GFR after cardiac surgery and in septic patients²²⁻³².

Currently, PENK data in children are lacking. In the pediatric population, biomarker research should focus on identifying age-adjusted reference values³³, since renal function in children is rapidly changing, especially in the first year of life⁹. Therefore, it is important to know the effect of age on PENK concentrations. The aim of this study is to determine reference values for PENK in healthy children from 0-1 years of age, identify changes in PENK concentrations during AKI in critically ill children and to compare PENK to other plasma biomarkers for GFR and AKI.

Materials & Methods

Overview

This prospective cohort study is part of a research project on AKI biomarkers in critically ill children (Sophia Foundation for Scientific Research, grant number 633)³⁴⁻³⁷. The main aim of the project is to establish reference values for AKI biomarkers in healthy children from 0-1 years and identify their predictive capabilities for AKI in critically ill children.

Setting

Healthy, full-term children without (pre)renal pathologies and critically ill children on mechanical ventilatory support from 0-1 years of age were included. Those with pre-existing kidney disease, rapidly expected death or ECMO treatment were excluded. Details on inclusion and exclusion criteria, collected patient data, sample collection and sample analysis can be found previous publications³⁴⁻³⁷. The study was approved by the Erasmus MC Medical Ethics Board. Deferred informed consent was obtained from parents and/or caregivers of all study subjects.

Clinical Patient Data

Demographic parameters (gender, diagnosis, postnatal age, ethnicity, weight) were collected for each subject. In addition, gestational age, birth weight, severity of illness scores (Pediatric Risk of Mortality [PRISM-III] score and Pediatric Index of Mortality [PIM-II] score), mechanical ventilation, vasopressor treatment, length of stay and mortality were collected for critically ill patients.

Sample Collection and Analysis

Blood samples were obtained from an indwelling arterial line or by capillary or venous puncture. In healthy children at least 1 sample was obtained before surgery or other medical procedures. In critically ill patients multiple blood samples were obtained up to 7 days after inclusion. Plasma samples were measured for SCr (enzymatic assay), CysC (immunoassay) and BTP (protein assay) in previous studies³⁴⁻³⁶. PENK was measured using a commercially available, double monoclonal sandwich immunoassay (sphingotec GmbH, Hennigsdorf, Germany)³⁸.

AKI Diagnosis

Healthy children were regarded as not having AKI, which was controlled by age-adjusted SCr z-scores, adapted from literature⁹. SCr z-scores between −2 and +2 were considered normal, patients with z-scores outside this range were excluded.

Critically ill patients were categorized into two subgroups (AKI and non-AKI) according to their highest attained RIFLE score within 48 hours of intubation³⁹, based on age-adjusted SCr reference values⁹. Patients with <150% increase of SCr were defined as non-AM whereas patients with 150-200%, 200-300% and >300% increase were categorized as Risk, Injury or Failure, respectively³⁹. Patients without a plasma sample of sufficient volume for PENK analysis or no sample within 48 hours of intubation were excluded. Additionally, as renal function can change during admission, mixed model and receiver-operating-characteristic (ROC) analyses were performed using the RIFLE score at time of blood sampling in order to account for the dynamic renal function in critically ill patients.

Statistical Analysis

Continuous data are presented as mean or median values with 95% confidence intervals (CI) values or interquartile ranges (IQR), depending on their distribution pattern. Categorical variables are presented as fractions with percentages (%).

Reference values of PENK were calculated in healthy children using the Generalized Additive Models for Location, Scale and Shape (GAMLSS) package in R⁴⁰. Akaike Information Criterion (AIC) values were used to determine the optimal model for these reference values and to identify age-related patterns in PENK concentrations. These reference values were converted to z-scores that follow a normal distribution, which were subsequently used for mixed model analyses in the critically ill cohort.

For critically ill children, baseline and clinical characteristics were compared between AKI and non-AM subgroups using Mann-Whitney U tests for continuous variables and Pearson's chi-square test for categorical variables. Categorical data with multiple categories were compared using the Kruskal-Wallis test with a post-hoc Dunn's test with Bonferroni-adjusted p-values to assess differences between categories.

Mixed Model Analysis

Critically ill patients had a variable number of samples with PENK concentrations. Therefore, a linear mixed model analysis was used to account for repeated measurements and independent variables using all samples during the whole study period. This analysis was used to determine whether PENK differed significantly between healthy and critically ill children with different severity of AKI. The dependent variable (PENK concentration) was transformed to z-scores to ensure an approximately normal distribution of model residuals. The independent variables in this model were RIFLE score at time of blood sampling, gender, postnatal age, time after intubation, vasopressor use and diagnosis categories based on their possible influence on the dependent variable. A random intercept was included to account for within-subject correlations. The results are presented using the estimated marginal means, which are the predicted values of the outcome (PENK z-score) adjusted for missing data and the effects of the independent variables. These estimated marginal means are back-transformed using the inverse of the z-score formula, producing an estimated prediction of median PENK values and its 95% CI. Estimated marginal means of PENK z-score in critically ill children were compared with healthy children using Student's T-test.

Explorative Analysis—Association of PENK and Other Biomarkers with AKI Diagnosis

Receiver operating characteristic (ROC) curves were used to evaluate the association of biomarker concentrations with AKI, in different time frames after intubation (0-6 hours, 6-12 hours, 12-24 hours, 24-36 hours and 36-48 hours). For this analysis, only the first sample per patient within each time frame was included to correct for repeated sampling, excluding missing values. The area under the ROC curve (AUROC) and its 95% CI were determined for PENK, CysC and BTP. Since AKI diagnosis was based on SCr, this biomarker was not included.

Statistical analyses were performed using SPSS statistics version 25.0 (IBM, Chicago, Ill., USA), GraphPad Prism version 5.03 (GraphPad Software Inc., La Jolla, Calif., USA) and R version 3.5.1 (R Core Team 2013). A two-sided p-value of 0.05 was considered the limit of statistical significance.

Results

Healthy Children

Out of 116 healthy children initially enrolled, 16 were excluded because no sufficient plasma sample was available for PENK measurements or SCr z-scores were outside the predefined range. Patient characteristics of the 100 remaining children are presented in Table 1.

There was no clear association between PENK and age during the first year in healthy children. The GAMLSS models including age as a covariate performed only marginally better than those without age, as indicated by AIC differences of only 0.952. Therefore, we used a GAMLSS model that assumes PENK concentrations were normally distributed after a Box-Cox transformation during the first year. PENK z-scores were calculated with the following formula that was derived from the GAMLSS model:

$$PENKz - score = \frac{{( \frac{P E N K}{517.34} )}^{- 0.01748} - 1}{- 0.01748 * {e}^{- 1.074}}$$

PENK concentrations were measured in 145 samples in total. Median PENK concentrations derived from this were 517.3 pmol/L (95% CI 488.9-547.4, 2.5^{th} and 97.5^{th} percentile at 265.2 and 1017.1 pmol/L, respectively), as shown in FIG. 2.

Critically Ill Children—Patient Characteristics

Of the 101 critically ill children in the previous studies, 10 were excluded: six patients had insufficient plasma volume for PENK analysis and four patients had no samples within 48 hours after intubation, leaving 91 critically ill children. Patient characteristics for all patients, AKI and non-AM subgroups are shown in Table 6.

Within 48 hours after intubation, 40 patients (44%) were categorized in the AKI subgroup; 20, 11 and 9 patients with Risk, Injury and Failure as their highest RIFLE score, respectively. There were no significant differences in gender, age, ethnicity and weight between non-AM and AKI subgroups. Disease severity scores, length of stay and mortality were higher in the AKI subgroup, but differences were not statistically significant (Table 6).

TABLE 6
Patient characteristics for healthy and critically ill cohorts, including subgroup analysis between AKI and non-AKI subgroups.

| | | | | |
|---|---|---|---|---|
| Healthy | Critically ill | Critically ill | Critically ill | P-value |
| children | All patients | Non-AKI^{b} | AKI^{b} | non-AKI vs |
| (n = 100) | (n = 91) | (n = 51) | (n = 40) | AKI |

Baseline characteristics

| Gender: male; n | 66/100 | (66%) | 59/91 | (64.8%) | 33/51 | (64.7%) | 26/40 | (65.0%) | 0.977 |
|---|---|---|---|---|---|---|---|---|---|
| Gestational age: weeks; | ^{a} | | 39.0 | (37.6-40.0) | 39.1 | (38.0-40.0) | 38.9 | (37.4-40.0) | 0.454 |
| median (IQR) | | | missing: 2 | | | | missing: 2 | | |
| Birth weight: kg; | ^{a} | | 3.1 | (2.8-3.6) | 3.2 | (2.9-3.6) | 3.1 | (2.8-3.6) | 0.666 |
| median (IQR) | | | missing: 6 | | missing: 2 | | missing: 4 | | |
| Ethnicity: Caucasian; n | 81/100 | (81%) | 64/91 | (70.3%) | 33/51 | (64.7%) | 31/40 | (77.5%) | 0.185 |

Clinical characteristics at admission

| | | | | | | | | | |
|---|---|---|---|---|---|---|---|---|---|
| Postnatal age: days; | 89.1 | (57.4-165.9) | 25.8 | (0.9-76.6) | 31.1 | (0.9-61.6) | 8.0 | (0.7-99.0) | 0.631 |
| median (IQR) | | | | | | | | | |
| Weight: kg; | 5.0 | (4.0-6.8) | 3.8 | (3.1-5.3) | 3.7 | (3.4-5.2) | 3.8 | (3.0-5.8) | 0.795 |

| | |
|---|---|
| median (IQR) | missing: 3 |

Diagnosis healthy cohort: n

| | | | |
|---|---|---|---|
| Inguinal hernia repair | 47/98 | (48%) | — |
| Orthopaedic surgery | 13/98 | (13%) | |
| Bronchoscopy | 9/98 | (9%) | |
| Hyperbilirubinemia | 7/98 | (7%) | |
| Sleep apnea test | 6/98 | (6%) | |
| Other | 16/98 | (16%) | |

missing: 2

Diagnosis critically ill cohort: n

| | | | | | | | |
|---|---|---|---|---|---|---|---|
| Respiratory failure | 32/91 | (35%) | 19/51 | (37%) | 13/40 | (33%) | 0.329 |
| CDH | 23/91 | (25%) | 12/51 | (24%) | 11/40 | (27.5%) | |
| Cardiac failure | 14/91 | (15%) | 5/51 | (10%) | 9/40 | (23%) | |
| Sepsis | 14/91 | (15%) | 9/51 | (18%) | 5/40 | (13%) | |
| Other critical illness | 8/91 | (9%) | 6/51 | (12%) | 2/40 | (5%) | |

Severity of illness:

| PIM-II predicted | ^{a} | 10.0 | (3.7-19.5) | 7.8 | (2.3-16.9) | 12.6 | (4.1-30.0) | 0.054 |
|---|---|---|---|---|---|---|---|---|
| mortality: %; | | missing: 6 | | missing: 2 | | missing: 4 | | 0.107 |
| median (IQR) | | 34.5 | (15.7-64.6) | 33.8 | (11.0-54.6) | 41.8 | (19.5-75.0) | |

| | | | |
|---|---|---|---|
| PRISM-III score: points; | missing: 6 | missing: 2 | missing: 4 |
| median (IQR) | | | |

Outcomes

| Duration of mechanical | ^{a} | | 5.8 | (3.1-11.0) | 5.3 | (3.1-9.1) | 6.8 | (3.1-18.2) | 0.095 |
|---|---|---|---|---|---|---|---|---|---|
| ventilation: days; | | | | | | | | | |
| median (IQR) | | | | | | | | | |
| Length of ICU stay: days; | ^{a} | | 9.7 | (6.2-22.2) | 8.4 | (5.9-15.6) | 16.2 | (7.0-31.9) | 0.077 |
| median (IQR) | | | | | | | | | |
| Hospital mortality: n | ^{a} | | 17/91 | (18.7%) | 8/51 | (15.7%) | 9/40 | (23%) | 0.408 |
| ICU mortality | | | 14/91 | (15.4%) | 6/51 | (11.8%) | 8/40 | (20%) | 0.446 |
| Initial PENK | 584.0 | (444.4-700.5) | 502.4 | (367.7-754.1) | 416.5 | (316.4-557.6) | 669.4 | (434.3-982.3) | <0.001 |
| concentration: pmol/L; | | | | | | | | | |
| median (IQR)^{c} | | | | | | | | | |
| Initial SCr z-score for age: | −0.08 | (−0.71-0.64) | 0.31 | (−0.58-1.70) | −0.42 | (−1.0-0.21) | 1.83 | (0.92-3.1) | <0.001 |
| median (IQR)^{c} | | | | | | | | | |

AKI = acute kidney injury; CDH = congenital diaphragmatic hernia; ICU = intensive care unit; IQR = interquartile range; PENK = proenkephalin A 119-159; PIM = pediatric index of mortality score; PRISM = pediatric risk of mortality score; SCr = serum creatinine concentration.
P-values show the differences between non-AKI and AKI subgroups in the critically ill cohort
^{a}: not collected in healthy cohort
^{b}: Critically ill children were stratified in AKI and non-AKI subgroups according to the highest attained RIFLE score within 48 hours after intubation
^{c}: Initial values represent the first sample after intubation that was available for analysis

Critically Ill Children—PENK Concentrations

A total of 578 plasma samples were analyzed for PENK in critically ill children. Median (IQR) initial PENK concentrations in the non-AM subgroup were 416.5 (316.4-557.6) pmol/L compared to 669.4 (434.3-982.3) pmol/L in patients with AKI (p<0.001). For the time frames up to 48 hours after intubation, PENK concentrations were significantly higher in the AKI subgroup (p<0.01 for every time frame, except for 36-48 hours after intubation (p=0.123)) (FIG. 3).

Critically Ill Children—Mixed Model Analysis

Of the included covariates in the final model, only age at showed a significant effect on PENK concentrations. An overview of the predicted values (back-transformed estimated marginal means) of PENK in critically ill children is depicted in FIG. 4, where they are compared to concentrations in healthy children. Median PENK concentrations were significantly lower among critically ill children without AKI (432.2 pmol/L 195% CI 398.2-469.21) compared to healthy children (517.3 pmol/l 195% CI 488.9-547.41, p<0.001). Critically ill patients with AKI had significantly higher median PENK concentrations than healthy children (except for patients with Risk as their highest RIFLE score) and critically ill children without AKI (Risk 507.9 pmol/L [95% CI 454.3-567.9], Injury 704.0 pmol/L [595.8-832.3] and Failure 930.5 pmol/L [763.2-1135.2], p<0.01 between all subgroups except Risk vs. Healthy children (p=0.746)).

Critically Ill Children—Correlation with Other Biomarkers

In order to compare the association of PENK and the other biomarkers to AKI diagnosis, ROC curves were generated in five-time frames after intubation (FIG. 5A-E). The AUROC for AKI by PENK was the highest in all but one timeframe up to 48 hours after intubation, with CysC having the second highest association with AKI. Only in the 12-24 hour time frame, PENK showed a lower AUROC than BTP. A list of AUROC values, 95% CIs and number of samples for each biomarker is shown in Table 7.

TABLE 7
Area under the receiver operating characteristic curves (AUROC)
with 95% confidence interval (CI) and number of samples for PENK,
cystatin C (CysC) and β-trace protein (BTP) in different time
frames after intubation for the association with acute kidney
injury at the time of sampling. Biomarkers are ranked top to
bottom from highest to lowest AUROC in each time frame.

| | | AUROC | No AKI | AKI samples |
|---|---|---|---|---|
| | Biomarker | (95% CI) | samples (n) | (n) |
| 0-6 hours | PENK | 0.843 (0.707- | 27 | 12 |
| after | | 0.979) | | |
| intubation | CysC | 0.702 (0.516- | 25 | 12 |
| | | 0.887) | | |
| | BTP | 0.598 (0.398- | 25 | 11 |
| | | 0.798) | | |
| 6-12 hours | PENK | 0.914 (0.813- | 22 | 10 |
| after | | 1.000) | | |
| intubation | CysC | 0.882 (0.761- | 19 | 10 |
| | | 1.000) | | |
| | BTP | 0.807 (0.606- | 14 | 5 |
| | | 1.000) | | |
| 12-24 hours | BTP | 0.881 (0.744- | 13 | 11 |
| after | | 1.000) | | |
| intubation | PENK | 0.859 (0.747- | 28 | 19 |
| | | 0.971) | | |
| | CysC | 0.805 (0.670- | 26 | 16 |
| | | 0.941) | | |
| 24-36 hours | PENK | 0.771 (0.653- | 41 | 23 |
| after | | 0.889) | | |
| intubation | CysC | 0.753 (0.612- | 34 | 18 |
| | | 0.893) | | |
| | BTP | 0.739 (0.571- | 22 | 15 |
| | | 0.908) | | |
| 36-48 hours | PENK | 0.603 (0.432- | 47 | 11 |
| after | | 0.775) | | |
| intubation | BTP | 0.586 (0.157- | 29 | 3 |
| | | 1.000) | | |
| | CysC | 0.544 (0.314- | 36 | 6 |
| | | 0.774) | | |

### DISCUSSION

We present the first data for PENK as a biomarker for AKI in children. In adults this biomarker has shown promising results, correlating strongly with GFR, AKI and deterioration of renal function^{22, 24, 26, 28}. In this study we present reference values for this biomarker in healthy children from 0-1 years of age. Despite the very high concentrations in children, PENK concentrations clearly discriminate between critically ill children with and without AKI. Moreover, our data suggest that it may outperform other frequently used AKI biomarkers.

In pediatric biomarker development, age-related reference values are crucial for their implementation, as ignoring age-dependent changes in normal values may lead to inaccurate performance in children³³. In our study this importance is apparent, as reference values for PENK in children up to 1 year of age are over ten-fold higher than in healthy adults (median 45 pmol/L, 99^{th} percentile at 80 pmol/L)²⁶. Furthermore, our reference values of PENK did not show an age-dependent change during the first year, in contrast to SCr⁹ and (to lesser extent) CysC^{16, 41}. Nevertheless, when comparing reference values of PENK between adults and children it is apparent PENK concentrations will decrease during childhood, as was also seen in our previous study for BTP³⁴ that showed (slightly) higher reference values throughout the first year compared to adults⁴².

These higher concentrations could be explained by a lower (absolute) clearance in young children, due to maturation of kidney function throughout the first years of life⁴³. However, since the GFR of young children is not ten-fold lower than the GFR of healthy adults, production of enkephalins might be increased in children. Concentrations of Met-enkephalin, another endogenous opioid derived from the same precursor as PENK, were also over ten-fold higher in children than in adults⁴⁴. Additionally, higher PENK concentrations in cerebrospinal fluid (CSF) were seen in 12 pediatric Moya-Moya Disease patients (aged 1-16), although concentrations in CSF did not reflect serum PENK concentrations⁴⁵.

Despite these higher median PENK concentrations in healthy children, its association with AKI in critically ill children is remarkable. This is in concordance with reports in adults, where PENK highly correlated with measured GFR in 24 septic intensive care patients²⁵ and was independently associated with RIFLE stages in another cohort of 101 sepsis patients²⁶. In our study PENK concentrations were also significantly elevated as AKI was more severe, even when correcting for repeated sampling and covariates. As this is the first data of PENK as a biomarker for AKI in children the comparison with other biomarkers is crucial. Compared to other biomarkers PENK showed a superior correlation to GFR compared to creatinine clearance in adults (R²0.91 vs. 0.68, respectively)²⁵, and a higher predictive value for AKI than NGAL (AUROC 0.73 vs. 0.68, respectively)²⁴. In children CysC and BTP are regarded among the best biomarkers for the diagnosis of AKI^{41, 46, 47} but in our exploratory analysis PENK showed the highest association with AKI. Additionally, the high association in this population with relatively mild AKI might suggest a higher sensitivity of PENK compared to CysC, making it a more suitable biomarker for early AKI screening. These promising findings warrant further investigation in the diagnostic potential of PENK in pediatric AKI.

Interestingly, PENK concentrations were lower in critically ill patients without AKI when compared to the reference values for healthy children. This might be explained by several pathophysiological processes during critical illness. Renal clearance can be elevated due to increased cardiac output and renal perfusion, a phenomenon called ‘augmented renal clearance’, which is well documented in adults⁴⁸ and children⁴⁹. Furthermore, extensive fluid challenges and edema might dilute PENK concentrations in plasma of critically ill patients. The fact that this pattern was seen for all other plasma biomarkers in our study (SCr, CysC and BTP) acknowledges this (data not shown).

In this study we used AKI in the first 48 hours after intubation as outcome measure, in contrast to 48 hours after admission, as multiple patients were not directly recruited and only had PENK concentrations days after admission. This resulted in slightly more AKI patients than previously reported³⁶. These additional patients mostly showed a mild and temporary increase of SCr. This increased representation of mild AKI patients could have resulted in less-pronounced differences in hard endpoints between AKI and non-AM subgroups than our group and others have shown when using AKI after admission as outcomes^{4-6, 36}.

Our study also has some limitations. The cohorts included children up to 1 year, which limits the extrapolation to other pediatric age groups. In addition, we could not validate PENK as a biomarker of GFR in children or the predictive capabilities for AKI, since GFR formulas for children using SCr and/or CysC have not been validated in children under 1 year^{50, 51}, gold standard measurements for GFR were not performed and the majority of patients showed signs of AKI before intubation³⁶. Lastly, detailed information on the urine output was unavailable from hospital records, which could have allowed for a more robust diagnosis of AKI by using KDIGO-criteria⁵². Although our results of PENK as a new biomarker for AKI are promising, future research regarding PENK in children should focus on identifying its reference values across the whole pediatric age range to elucidate any (long-term) age-related patterns. Furthermore, additional comparisons between measured GFR and PENK concentrations should be performed in (critically ill) children, as is done in adults^{23, 27, 38}, in order to establish PENK as a new biomarker for GFR and AKI in this population.

### CONCLUSION

This research represents the first data of PENK as a biomarker for AKI in children. PENK concentrations appear to be stable during the first year of life, but show great inter-individual variability, with reference values considerably higher in children than adults. Regardless, results of this biomarker for diagnosing AKI in children are very promising. PENK showed the strongest association with AKI diagnosis, outperforming other plasma biomarkers that are regarded as the best currently available. Ongoing research must validate whether this biomarker, alone or in combination with others, has the potential to improve the diagnosis and treatment of children with AKI.

### REFERENCES

1. McGregor T L, Jones D P, Wang L, et al. Acute Kidney Injury Incidence in Noncritically Ill Hospitalized Children, Adolescents, and Young Adults: A Retrospective Observational Study. Am J Kidney Dis. 2016; 67(3):384-390.
2. Sutherland S M, Kwiatkowski D M. Acute Kidney Injury in Children. Adv Chronic Kidney Dis. 2017; 24(6):380-387.
3. Lameire N, Van Biesen W, Vanholder R. Epidemiology of acute kidney injury in children worldwide, including developing countries. Pediatr Nephrol. 2017; 32(8):1301-1314.
4. Alkandari O, Eddington K A, Hyder A, et al. Acute kidney injury is an independent risk factor for pediatric intensive care unit mortality, longer length of stay and prolonged mechanical ventilation in critically ill children: a two-center retrospective cohort study. Crit Care. 2011; 15(3):R146.
5. Kaddourah A, Basu R K, Bagshaw S M, Goldstein S L, Investigators A. Epidemiology of Acute Kidney Injury in Critically Ill Children and Young Adults. N Engl J Med. 2017; 376(1): 11-20.
6. Goldstein S L. Acute kidney injury in children and its potential consequences in adulthood. Blood Purif. 2012; 33(1-3):131-137.
7. Soveri I, Berg U B, Bjork J, et al. Measuring GFR: a systematic review. Am J Kidney Dis. 2014; 64(3):411-424.
8. Guignard J P, Drukker A. Why do newborn infants have a high plasma creatinine? Pediatrics. 1999; 103(4):e49.
9. Boer D P, de Rijke Y B, Hop W C, Cransberg K, Dorresteijn E M. Reference values for serum creatinine in children younger than 1 year of age. Pediatr Nephrol. 2010; 25(10):2107-2113.
10. Gross J L, Prowle J R. Perioperative acute kidney injury. BJA Education. 2015; 15(4):213-218.
11. Inker L A, Tighiouart H, Coresh J, et al. GFR Estimation Using beta-Trace Protein and beta2-Microglobulin in CKD. Am J Kidney Dis. 2016; 67(1):40-48.
12. Schwartz G J, Schneider M F, Maier P S, et al. Improved equations estimating GFR in children with chronic kidney disease using an immunonephelometric determination of cystatin C. Kidney Int. 2012; 82(4):445-453.
13. Deng F, Finer G, Haymond S, Brooks E, Langman C B. Applicability of estimating glomerular filtration rate equations in pediatric patients: comparison with a measured glomerular filtration rate by iohexol clearance. Transl Res. 2015; 165(3):437-445.
14. Ashraf M, Shahzad N, Irshad M, Hussain S Q, Ahmed P. Pediatric acute kidney injury: A syndrome under paradigm shift. Indian J Crit Care Med. 2014; 18(8): 518-526.
15. Buijs E A, Zwiers A J, Ista E, Tibboel D, de Wildt S N. Biomarkers and clinical tools in critically ill children: are we heading toward tailored drug therapy? Biomark Med. 2012; 6(3):239-257.
16. Bokenkamp A, Domanetzki M, Zinck R, Schumann G, Brodehl J. Reference values for cystatin C serum concentrations in children. Pediatr Nephrol. 1998; 12(2):125-129.
17. Vynckier L L, Flore K M, Delanghe S E, Delanghe J R. Urinary beta-trace protein as a new renal tubular marker. Clin Chem. 2009; 55(6):1241-1243.
18. Mahmoodpoor A, Hamishehkar H, Fattahi V, Sanaie S, Arora P, Nader N D. Urinary versus plasma neutrophil gelatinase-associated lipocalin (NGAL) as a predictor of mortality for acute kidney injury in intensive care unit patients. J Clin Anesth. 2018; 44:12-17.
19. Ernst A, Kohrle J, Bergmann A. Proenkephalin A 119-159, a stable proenkephalin A precursor fragment identified in human circulation. Peptides. 2006; 27(7):1835-1840.
20. Denning G M, Ackermann L W, Barna T J, et al. Proenkephalin expression and enkephalin release are widely observed in non-neuronal tissues. Peptides. 2008; 29(1):83-92.
21. Zoccali C, Ciccarelli M, Mallamaci F, Maggiore Q, Lotti M, Zucchelli G C. Plasma met-enkephalin and leu-enkephalin in chronic renal failure. Nephrol Dial Transplant. 1987; 1(4):219-222.
22. Beunders R S J, Wu A, Zarbock A, Di Somma S, Mehta R. Pickkers P. Proenkephalin (PENK) as a Novel Biomarker for Kidney Function. Journal of Applied Laboratory Medicine. 2017; 2(3):400-412.
23. Kieneker L M, Hartmann O, Struck J, et al. Plasma Proenkephalin and Poor Long-Term Outcome in Renal Transplant Recipients. Transplant Direct. 2017; 3(8):e190.
24. Kim H, Hur M, Lee S, et al. Proenkephalin, Neutrophil Gelatinase-Associated Lipocalin, and Estimated Glomerular Filtration Rates in Patients With Sepsis. Ann Lab Med. 2017; 37(5):388-397.
25. Leijte G, Beunders R, Van Groenendael R, Peters E, Kox M, Pickkers P. Proenkephalin, the new marker for kidney function on the Intensive care unit? Intensive Care Medicine Experimental Conference: 30th Annual Congress of the European Society of Intensive Care Medicine, ESICM. 2017; 5(2 Supplement 1).
26. Marino R, Struck J, Hartmann O, et al. Diagnostic and short-term prognostic utility of plasma pro-enkephalin (pro-ENK) for acute kidney injury in patients admitted with sepsis in the emergency department. Journal of nephrology. 2015; 28(6):717-724.
27. Matsue Y, Ter Maaten J M, Struck J, et al. Clinical Correlates and Prognostic Value of Proenkephalin in Acute and Chronic Heart Failure. J Card Fail. 2017; 23(3):231-239.
28. Ng L L, Squire I B, Jones D J, et al. Proenkephalin, Renal Dysfunction, and Prognosis in Patients With Acute Heart Failure: A GREAT Network Study. J Am Coll Cardiol. 2017; 69(1):56-69.
29. Schulz C A, Christensson A, Ericson U, et al. High Level of Fasting Plasma Proenkephalin-A Predicts Deterioration of Kidney Function and Incidence of CKD. J Am Soc Nephrol. 2017; 28(1):291-303.
30. Shah K S, Taub P, Patel M, et al. Proenkephalin predicts acute kidney injury in cardiac surgery patients. Clin Nephrol. 2015; 83 (1): 29-35.
31. Siong Chan D C, Cao T H, Ng L L. Proenkephalin in Heart Failure. Heart Fail Clin. 2018; 14(1):1-11.
32. Hollinger A, Wittebole X, Francois B, et al. Proenkephalin A 119-159 (Penkid) Is an Early

Biomarker of Septic Acute Kidney Injury: The Kidney in Sepsis and Septic Shock (Kid-SSS) Study. Kidney Int Rep. 2018; 3(6):1424-1433.
33. Greenberg J H, Parikh C R. Biomarkers for Diagnosis and Prognosis of AKI in Children: One Size Does Not Fit All. Clin J Am Soc Nephrol. 2017; 12(9):1551-1557.
34. Zwiers A J, Cransberg K, de Rijke Y B, et al. Reference ranges for serum beta-trace protein in neonates and children younger than 1 year of age. Clin Chem Lab Med. 2014; 52(12):1815-1821.
35. Zwiers A J, de Wildt S N, de Rijke Y B, et al. Reference intervals for renal injury biomarkers neutrophil gelatinase-associated lipocalin and kidney injury molecule-1 in young infants. Clin Chem Lab Med. 2015; 53(8):1279-1289.
36. Zwiers A J, de Wildt S N, van Rosmalen J, et al. Urinary neutrophil gelatinase-associated lipocalin identifies critically ill young children with acute kidney injury following intensive care admission: a prospective cohort study. Crit Care. 2015; 19:181.
37. Zwiers A J, Cransberg K, de Rijke Y B, van Rosmalen J, Tibboel D, de Wildt S N. Urinary

Neutrophil Gelatinase-Associated Lipocalin Predicts Renal Injury Following Extracorporeal Membrane Oxygenation. Pediatr Crit Care Med. 2015; 16(7):663-670.
38. Donato L J, Meeusen J W, Lieske J C, Bergmann D, Sparwasser A, Jaffe A S. Analytical performance of an immunoassay to measure proenkephalin. Clin Biochem. 2018; 58:72-77.
39. Bellomo R, Ronco C, Kellum J A, Mehta R L, Palevsky P, Acute Dialysis Quality Initiative w. Acute renal failure—definition, outcome measures, animal models, fluid therapy and information technology needs: the Second International Consensus Conference of the Acute Dialysis Quality Initiative (ADQI) Group. Crit Care. 2004; 8(4):R204-212.
40. Rigby R A, Stasinopoulos, D. M. Generalized additive models for location, scale and shape. Appl Statis. 2005; 54(3):507-554.
41. Bai Z, Fang F, Xu Z, et al. Serum and urine FGF23 and IGFBP-7 for the prediction of acute kidney injury in critically ill children. BMC Pediatr. 2018; 18(1).
42. Pottel H, Schaeffner E, Ebert N. Evaluating the diagnostic value of rescaled beta-trace protein in combination with serum creatinine and serum cystatin C in older adults. Clin Chim Acta. 2018; 480:206-213.
43. Kearns G L, Abdel-Rahman S M, Alander S W, Blowey D L, Leeder J S, Kauffman R E. Developmental pharmacology—drug disposition, action, and therapy in infants and children. N Engl J Med. 2003; 349(12):1157-1167.
44. Martinez A M, Padbury J F, Burnell E E, Thio S L. Plasma methionine enkephalin levels in the human newborn at birth. Biol Neonate. 1991; 60(2):102-107.
45. Yokoyama K, Maruwaka M, Yoshikawa K, et al. Elevation of Proenkephalin 143-183 in Cerebrospinal Fluid in Moyamoya Disease. World Neurosurg. 2018; 109:e446-e459.
46. McCaffrey J, Coupes B, Chaloner C, Webb N J, Barber R, Lennon R. Towards a biomarker panel for the assessment of AKI in children receiving intensive care. Pediatr Nephroi. 2015; 30(10):1861-1871.
47. Safdar O Y, Shalaby M, Khathlan N, et al. Serum cystatin is a useful marker for the diagnosis of acute kidney injury in critically ill children: prospective cohort study. BMC Nephrol. 2016; 17(1):130.
48. Udy A A, Jarrett P, Stuart J, et al. Determining the mechanisms underlying augmented renal drug clearance in the critically ill: use of exogenous marker compounds. Crit Care. 2014; 18(6):657.
49. Dhont E, Van Der Heggen T, De Jaeger A, Vande Walle J, De Paepe P, De Cock P A. Augmented renal clearance in pediatric intensive care: are we undertreating our sickest patients? Pediatr Nephrol. 2018.
50. Bouvet Y, Bouissou F, Coulais Y, et al. GFR is better estimated by considering both serum cystatin C and creatinine levels. Pediatr Nephrol. 2006; 21(9):1299-1306.
51. Pottel H, Dubourg L, Goffin K, Delanaye P. Alternatives for the Bedside Schwartz Equation to Estimate Glomerular Filtration Rate in Children. Adv Chronic Kidney Dis. 2018; 25(1):57-66.
52. Kidney Disease Improving Global Outcomes (KDIGO): Clinical Practice Guideline for Acute Kidney Injury. Kidney Int Suppl. 2012; 2(1): 1-138.

### Example 3

PENK in Healthy Children

Healthy children without known kidney impairment (n=38, 55.3% female, mean [range] age 11.3 [1-20] years, SCr 49.7 [23-98] μmon) were measured using the MR-PENK assay.

PENK levels decreased age dependently (FIG. 6). There was a strong correlation between PENK and age (r=−0.86, p<0.0001).

### FIGURE DESCRIPTION

FIG. 1: A typical Pro-Enkephalin dose/signal curve.

FIG. 2: Reference percentiles for Proenkephalin A 119-159 (PENK) as a linear function of postnatal age in healthy children.

Squares represent all measured PENK concentrations (145 samples) of all healthy patients (n=100) in our healthy cohort. Reference percentiles for PENK are indicated at the 2.5th (dash-dotted, bottom), 25th (dashed, bottom), 50th (solid), 75th (dashed, top) and 97.5th (dash-dotted, top) percentiles.

FIG. 3: PENK concentrations in critically ill patients with AKI compared to non-AKI in the first 48 hours after intubation.

Plasma PENK levels presented as median and interquartile range, for 5 time-frames following intubation. Outcomes are stratified for ‘No AKI’ (green) or ‘AKI’ (red) within 48 hours after intubation, one sample per patient per time frame. Numbers in brackets above each time frame represent the number of patients in each category. **: p<0.01; ***: p<0.001; n.s: not statistically significant (p>0.05).

FIG. 4: PENK concentrations in healthy and critically ill children with different levels of AKI. Overview of median PENK concentrations with 95% confidence intervals in 100 healthy children (145 samples) and 91 critically ill children with different degrees of AKI at time of sampling based on RIFLE scores (561 total samples: 422 No AKI, 86 Risk, 28 Injury, 25 Failure). 17 PENK samples unused due to missing corresponding RIFLE scores.

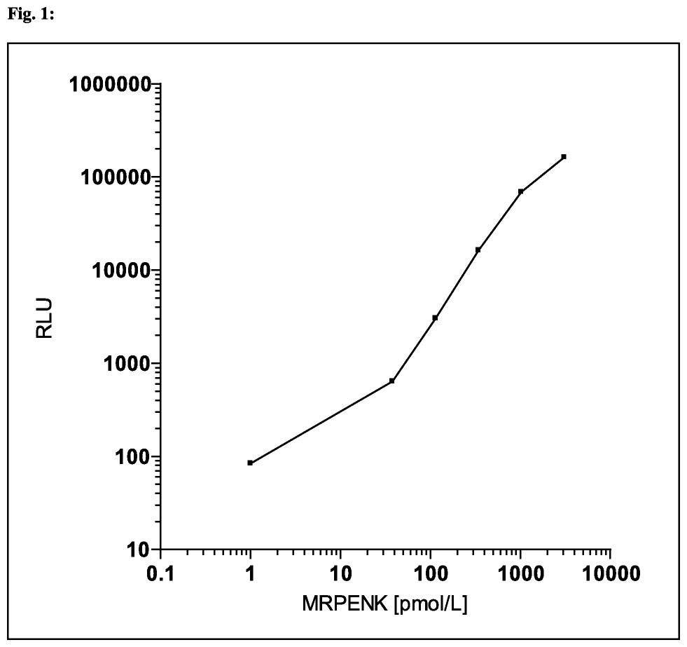

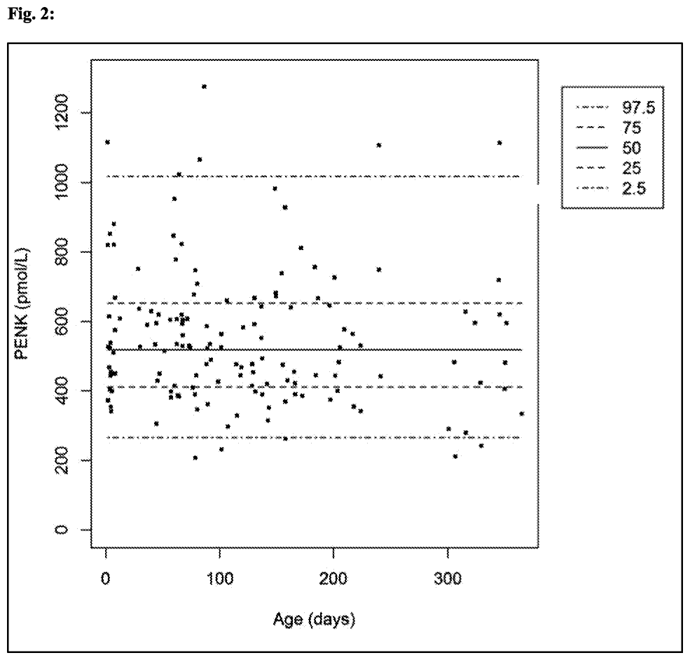

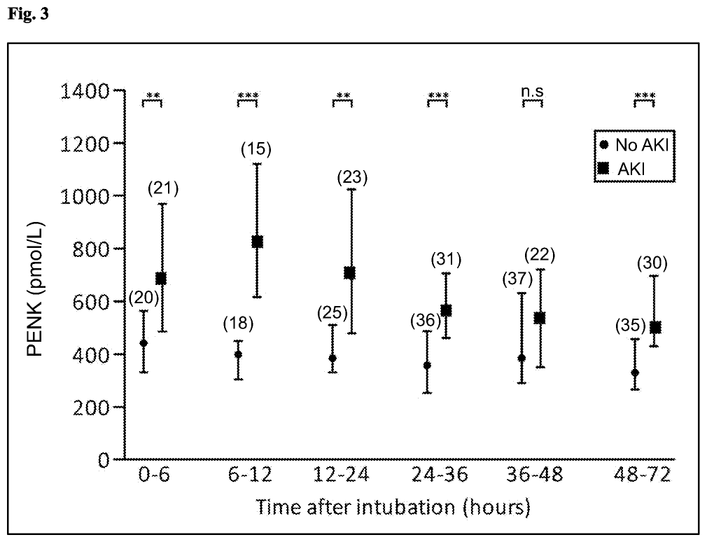

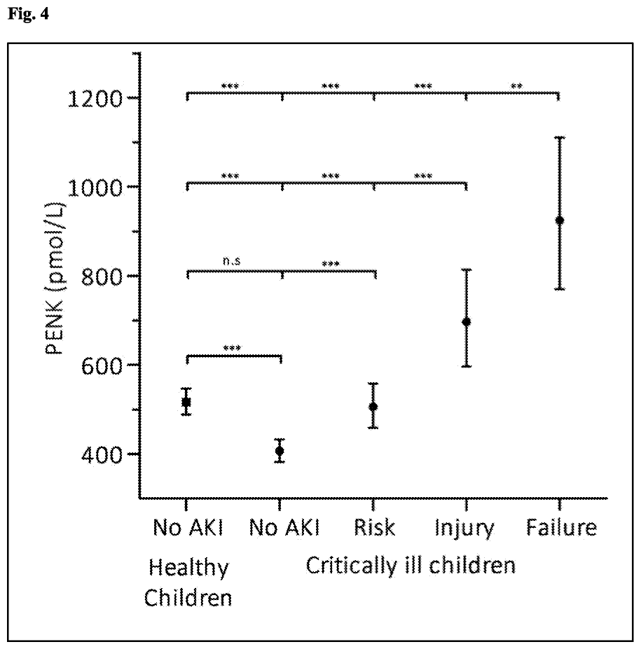

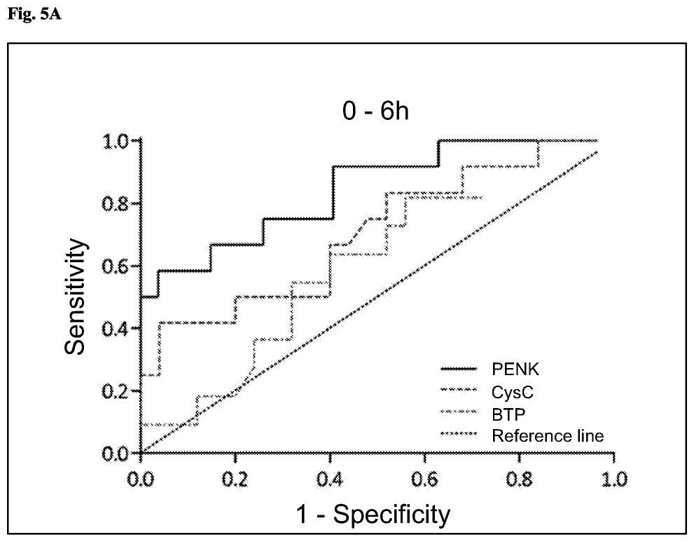

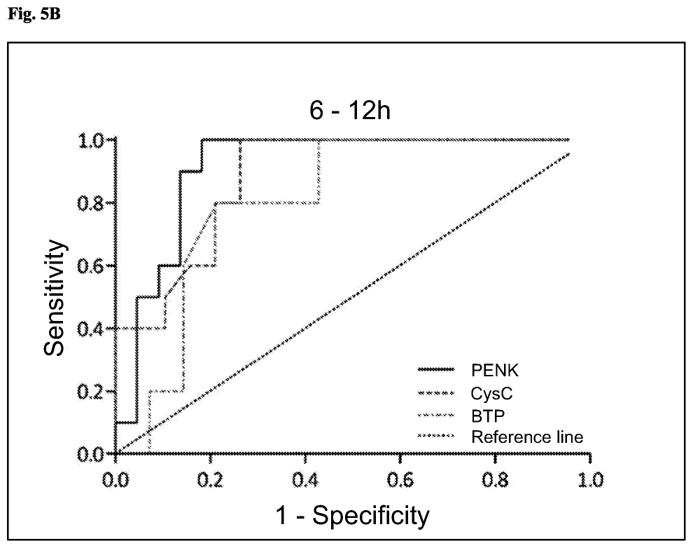

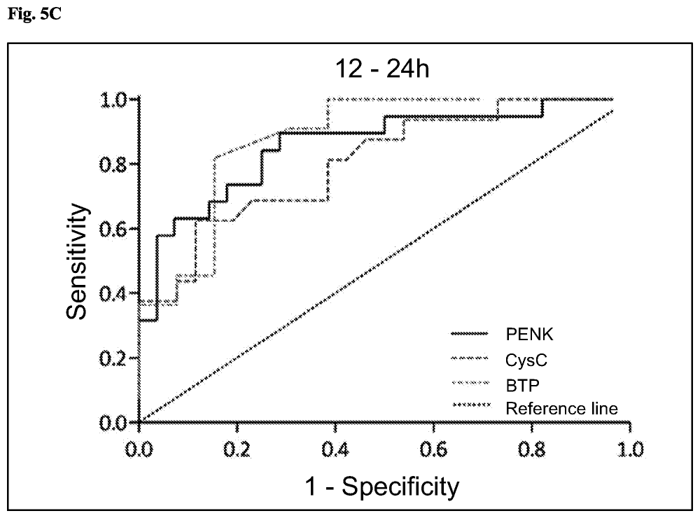

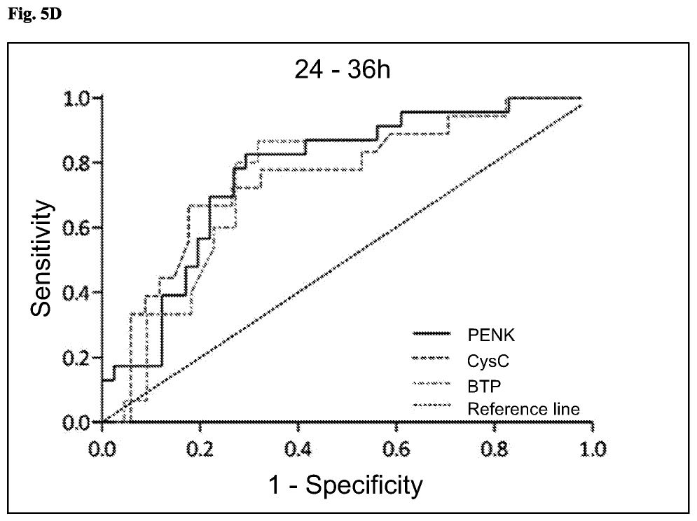

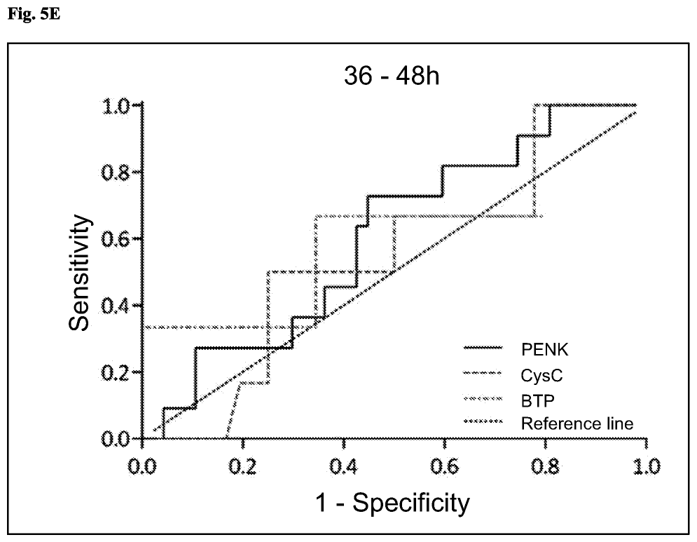

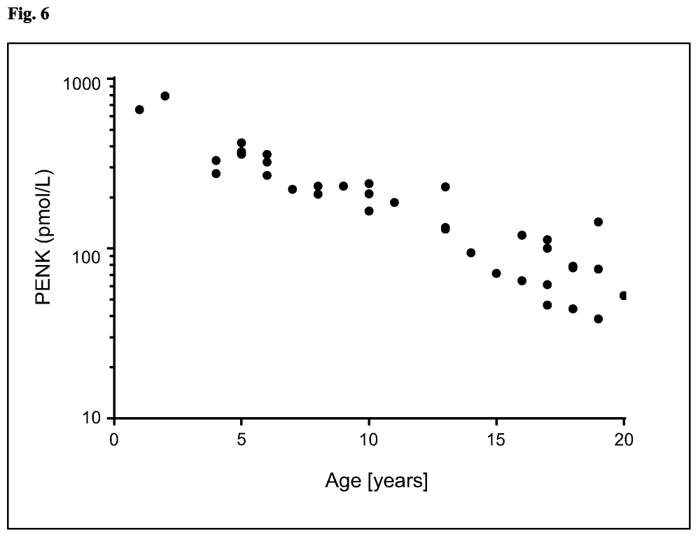

Statistical significance in critically ill children is based on results of estimated marginal means from the linear mixed model analysis, corrected for repeated sampling and all covariates (RIFLE score, gender, postnatal age, time after intubation, vasopressor use and diagnosis categories), using all samples during the whole study 91 critically ill children (561 samples) and the corresponding RIFLE stage at the time of blood sampling. Differences between estimated marginal means of critically ill children and the mean PENK concentrations of healthy children (145 samples) determined using students T-test. **: p<0.01; ***: p<0.001; n.s: not statistically significant (p>0.05)

FIG. 5A-E: Performance of PENK, cystatin C and BTP for AKI diagnosis. Receiver operating characteristic (ROC) curves for PENK, CysC and BTP for their association with AKI at the time of blood sampling in 5 timeframes: A (0-6 h), B (6-12 h), C (12-24 h), D (24-36 h) and E (36-48 h) after intubation. A list of AUROC values, confidence intervals and number of samples in each time frame for each biomarker is shown in Table 7.

FIG. 6: PENK concentrations in healthy children (up to 20 years of age).

1. A method for (a) diagnosing or monitoring kidney function in a subject or (b) diagnosing kidney dysfunction in a subject or (c) predicting or monitoring the risk of an adverse event in a diseased subject, wherein said adverse event is selected from the group comprising worsening of kidney function including kidney failure, loss of kidney function and end-stage kidney disease or death due to kidney dysfunction including kidney failure, loss of kidney function and end-stage kidney disease or (d) predicting or monitoring the success of a therapy or intervention comprising:
determining the level of Pro-Enkephalin or fragments thereof of at least 5 amino acids in a bodily fluid obtained from said subject; and
(a) correlating said level of Pro-Enkephalin or fragments thereof with kidney function in a subject or
(b) correlating said level of Pro-Enkephalin or fragments thereof with kidney dysfunction, wherein an elevated level above a certain threshold is predictive or diagnostic for kidney dysfunction in said subject or
(c) correlating said level of Pro-Enkephalin or fragments thereof with said risk of an adverse event in a diseased subject, wherein an elevated level above a certain threshold is predictive for an enhanced risk of said adverse events or
(d) correlating said level of Pro-Enkephalin or fragments thereof with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions,
wherein said Pro-Enkephalin or fragment is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11,
wherein said threshold is in the range of 150-1290 pmol/L,
and wherein said subject is a child.

2. A method for diagnosing or monitoring kidney function in a subject comprising:
determining the level of Pro-Enkephalin or fragments thereof of at least 5 amino acids in a bodily fluid obtained from said subject; and
wherein during follow-up measurement, a relative change of Pro-Enkephalin and fragments thereof that is lowered correlates with the improvement of the subject's kidney function, or
wherein during follow-up measurement, a relative change of Pro-Enkephalin and fragment thereof that is increased correlates with the worsening of the subject's kidney function,
wherein said Pro-Enkephalin or fragment thereof is selected from the group comprising SEQ ID No. 1, SEQ ID No. 2, SEQ ID No. 5, SEQ ID No. 6, SEQ ID No. 8, SEQ ID No. 9, SEQ ID No. 10 and SEQ ID No. 11; and
wherein said determination of Pro-Enkephalin or fragments thereof of at least 5 amino acids is performed more than once in one patient,
wherein the subject is a child.

3. A method according to claim 1, wherein said subject is a child at the age of 18 years or below, more preferred at the age of 14 years or below, even more preferred at the age of 12 years or below, even more preferred at the age of 8 years or below, even more preferred at the age of 5 years or below, even more preferred at the age of 2 years or below, most preferred at the age of one year or below.

4. A method according to claim 1, wherein said child is critically ill.

5. A method according to claim 1 comprising determining the level of immunoreactive analyte by using at least one binder that binds to a region within the amino acid sequence of Pro-Enkephalin (PENK) or fragments thereof in a bodily fluid obtained from said subject; and
(a) correlating said level of immunoreactive analyte with kidney function in a subject or
(b) correlating said level of immunoreactive analyte with kidney dysfunction wherein an elevated level above a certain threshold is predictive or diagnostic for kidney dysfunction in said subject or
(c) correlating said level of immunoreactive analyte with said risk of an adverse event in a diseased subject, wherein an elevated level above a certain threshold is predictive for an enhanced risk of said adverse events or
(d) correlating said level of immunoreactive analyte with success of a therapy or intervention in a diseased subject, wherein a level below a certain threshold is predictive for a success of therapy or intervention.

6. A method according to claim 1, wherein said at least one binder binds to a region within the amino acid sequence selected from the group comprising SEQ ID No. 1, 2, 5, 6, 8, 9, 10 and 11, preferably said at least one binder binds to a region with the sequences selected from the group comprising SEQ ID No. 1, 2, 5, 6, 8 and 9, preferably said at least one binder binds to SEQ ID No. 6.

7. A method according to claim 1, wherein the level of Pro-Enkephalin is measured with an immunoassay and said binder is an antibody, or an antibody fragment binding to Pro-Enkephalin or fragments thereof of at least 5 amino acids.

8. A method according to claim 1, wherein an assay is used comprising two binders that bind to two different regions within the region of Pro-Enkephalin that is amino acid 133-140 (LKELLETG, SEQ ID NO. 13) and amino acid 152-159 (SDNEEEVS, SEQ ID No. 14), wherein each of said regions comprises at least 4 or 5 amino acids.

9. A method according to claim 1, wherein an assay is used for determining the level of Pro-Enkephalin or fragments thereof of at least 5 amino acids and wherein the assay sensitivity of said assay is able to quantify the Pro-Enkephalin or Pro-Enkephalin fragments of healthy subjects and is <15 pmol/L.

10. A method according to claim 1, wherein said bodily fluid may be selected from the group comprising whole blood, serum, plasma, urine, cerebrospinal liquid (CSF), and saliva.

11. A method according to claim 1, wherein additionally at least one clinical parameter is determined selected from the group comprising: beta-trace protein (BTP), cystatin C, KIM-1, TIMP-2, IGFBP-7, blood urea nitrogen (BUN), NGAL, Creatinine Clearance, serum Creatinine (SCr), urea, Pediatric Risk of Mortality III [PRISM-III] score, Pediatric Index of Mortality 2 [PIM-II] score and Apache Score.

12. A method according to claim 1, wherein said determination of Pro-Enkephalin or fragments thereof of at least 5 amino acids is performed more than once in one patient.

13. A method according to claim 1, wherein said monitoring is performed in order to evaluate the response of said subject to preventive and/or therapeutic measures taken.

14. A method according to claim 1 in order to stratify said subjects into risk groups.

15. A point-of-care device for performing a method according to claim 1, wherein said point of care device comprises at least two antibodies or antibody fragments directed to amino acid 133-140 (LKELLETG, SEQ ID No. 13) and amino acid 152-159 (SDNEEEVS, SEQ ID No. 14).

16. A kit for performing a method according to claim 1, wherein said kit comprises at least two antibodies or antibody fragments directed to amino acid 133-140 (LKELLETG, SEQ ID No. 13) and amino acid 152-159 (SDNEEEVS, SEQ ID No. 14).
